(19) United States
(12) Patent Application Publication
(10) Pub. No.: US 2026/0237520 A1
(43) Pub. Date: Aug. 13, 2026

(54) SYSTEMS AND METHODS FOR ENVIRONMENTAL HEALTH RISK PREDICTION AND AUTOMATED PATIENT OUTREACH

(71) Applicant: Samrat V. Kulkarni, Hoboken, NJ (US)

(72) Inventor: Samrat V. Kulkarni, Hoboken, NJ (US)

(21) Appl. No.: 19/537,680

(22) Filed: Feb. 12, 2026

Related U.S. Application Data
(60) Provisional application No. 63/757,496, filed on Feb. 12, 2025.

(51) Int. Cl. G16H 50/30; G16H 10/60; G16H 50/70; G16H 70/20; G16H 70/40; G16H 70/60

(52) U.S. Cl. CPC G16H 50/30; G16H 10/60; G16H 50/70; G16H 70/20; G16H 70/40; G16H 70/60

(57) ABSTRACT

A computer-based system predicts patient hospitalization risk due to adverse environmental conditions and automates proactive patient outreach to prevent hospitalizations. The system extracts patient health data from medical claims systems, pharmacy claims systems, electronic health records, and care management systems. The system receives forecasted environmental data including heat index, air quality index, pollen count, and cold temperature values, and compares forecasted values to predefined thresholds. When thresholds are exceeded, the system calculates patient-specific risk scores by correlating patient chronic conditions and medication regimens with forecasted environmental conditions, including determining predicted reductions in medication effectiveness. The system generates differentiated outreach target lists based on risk severity and patient contact permissions, then deploys voice and text artificial intelligence agents to deliver personalized health guidance before adverse conditions occur. Interaction summaries documenting patient communications are stored in electronic health records.

### CROSS-REFERENCE TO RELATED APPLICATIONS

The present application claims priority to U.S. Provisional Application No. 63/757,496 filed Feb. 12, 2025, titled “AI DRIVEN SYSTEMS AND METHODS FOR PATIENT HEALTH RISK PREDICTION & HOSPITALIZATION PREVENTION DUE TO ENVIRONMENTAL FACTORS,” which is hereby incorporated by reference in its entirety.

### TECHNICAL FIELD

The embodiments generally relate to the technical field of computer-based healthcare risk prediction and patient outreach systems for preventing hospitalization due to adverse environmental conditions.

### BACKGROUND

Conventional healthcare management systems are designed to facilitate patient monitoring and care coordination across healthcare organizations. Such systems often operate as part of broader enterprise health platforms or as integrated modules within electronic health record software.

These systems typically support the storage and organization of patient medical histories, medication records, and care management encounters. Many employ reactive processes to address patient health concerns after symptoms manifest or after patients present at emergency departments. Environmental health alerts from governmental agencies such as weather services provide broad population-level warnings about heat waves, bad air quality, extreme cold weather events, and other environmental hazards, but these alerts are not customized based on individual patient health profiles or medication regimens.

Some healthcare organizations have implemented nurse outreach programs to contact at-risk patients during environmental events. However, given the temporal and unpredictable nature of environmental hazards, mobilizing trained human nurses to conduct proactive outreach can be expensive and logistically challenging. Human-based outreach programs face limitations in scalability, availability outside business hours, and speed of deployment when environmental conditions change rapidly.

Conventional solutions may also generate analytics summarizing patient populations at risk for various conditions. These analytics are often used by care managers to identify patients for intervention programs. In many cases, risk assessment within these systems is based on historical health data alone without integration of predicted environmental conditions, and outreach programs operate independently from risk prediction engines without automated triggering based on real-time or forecasted environmental data.

While such systems provide efficiencies in managing patient populations, they face challenges in correlating individual patient vulnerabilities with environmental forecasts, automating patient outreach based on integrated risk assessments, or scaling interventions rapidly when environmental conditions threaten patient health. Patient health records are often maintained in systems disconnected from environmental monitoring data sources, and medication effectiveness considerations under varying environmental conditions are not systematically incorporated into risk assessments.

Consequently, there is a need for an improved healthcare risk prediction and patient outreach system that addresses the limitations of reactive intervention approaches, reduces reliance on manual outreach processes, and provides enhanced integration between patient health data, environmental forecasting, and automated patient communication.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In one aspect, the disclosed system, method, or software product may include a data integration module configured to extract patient-level health data from medical claims databases, pharmacy claims databases, electronic health record systems, or care management systems. The module may retrieve patient medical histories, current medication regimens, chronic condition indicators, and care management encounter records through application programming interfaces or custom data extraction routines.

In one aspect, embodiments may include an environmental data module configured to receive forecasted environmental condition data from environmental data sources. The module may retrieve predicted values for heat indices, air quality indices, pollen counts, and cold temperatures for future time periods and may determine when forecasted environmental condition data exceeds predefined environmental thresholds.

In one aspect, embodiments may include a risk prediction engine configured to generate patient-specific hospitalization risk scores and risk factor breakdowns by correlating patient-level health data with forecasted environmental condition data. The engine may determine predicted reductions in medication effectiveness for current medication regimens when forecasted environmental conditions exceed predefined thresholds, and may identify relationships between chronic conditions and environmental vulnerabilities based on condition-specific susceptibility values derived from historical hospitalization data.

In one aspect, embodiments may include an orchestration engine configured to generate voice outreach target lists and text outreach target lists based on patient-specific hospitalization risk scores and patient contact permissions. The engine may compare risk scores to predefined risk thresholds and may apply permitted timing and permitted communication channel rules for contacting each patient.

In one aspect, embodiments may include an automated outreach module configured to initiate proactive patient communication via artificial intelligence agents. The module may deploy voice artificial intelligence agents to conduct spoken dialogues with patients and text artificial intelligence agents to conduct text-based messaging exchanges, wherein each agent delivers personalized health guidance based on the risk factor breakdown.

In one aspect, embodiments may include a clinical summarization engine configured to generate interaction summaries documenting proactive patient communications and to store the interaction summaries in electronic health record systems. The engine may record timestamps, communication channels used, patient responses received, and recommended follow-up actions.

In some aspects, the system may include at least one computing device in operable communication with a network and a server in operable communication with the network to host a patient risk management platform containing the above-described modules. The computing device may execute instructions to perform the operations described herein, thereby enabling automated, AI-driven patient outreach that prevents hospitalizations due to adverse environmental conditions.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 is a flow diagram illustrating an exemplary operational sequence of the Data Integration Module, according to some embodiments;

FIG. 4 is a flow diagram illustrating an exemplary operational sequence of the Environmental Data Module, according to some embodiments;

FIG. 5 is a flow diagram illustrating an exemplary operational sequence of the Risk Prediction Engine, according to some embodiments;

FIG. 6 is a flow diagram illustrating an exemplary operational sequence of the Orchestration Engine, according to some embodiments;

FIG. 7 is a flow diagram illustrating an exemplary operational sequence of the Automated Outreach Module, according to some embodiments;

FIG. 8 is a flow diagram illustrating an exemplary operational sequence of the Clinical Summarization Engine, according to some embodiments; and

FIG. 9 is a flow diagram illustrating an exemplary end-to-end system operational flow, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. As used herein, the terms “application” and “platform” may be used interchangeably to refer to the patient risk management platform and its associated software components.

The disclosed system may include at least one computing device in operable communication with a network and a server configured to host and execute a patient risk management platform. The patient risk management platform may include multiple functional modules, each implemented in software, firmware, hardware, or any combination thereof. In some embodiments, the modules may include a Data Integration Module configured to extract patient-level health data from medical and pharmacy claims systems, electronic health record systems, and care management systems via application programming interfaces or custom data extraction routines. An Environmental Data Module may receive forecasted environmental condition data from environmental data sources and determine when forecasted values for heat index, air quality index, pollen count, or cold temperature exceed predefined environmental thresholds for future time periods. A Risk Prediction Engine may generate patient-specific hospitalization risk scores and risk factor breakdowns by correlating patient-level health data with forecasted environmental condition data, including determining predicted reductions in medication effectiveness when environmental conditions exceed thresholds. An Orchestration Engine may generate voice outreach target lists and text outreach target lists by comparing patient-specific hospitalization risk scores to predefined risk thresholds and applying patient contact permissions that define permitted timing and permitted communication channels for contacting each patient. An Automated Outreach Module may initiate proactive patient communication via voice artificial intelligence agents and text artificial intelligence agents to deliver personalized health guidance based on risk factor breakdowns. A Clinical Summarization Engine may generate interaction summaries documenting proactive patient communications and store the interaction summaries in electronic health record systems. A Communication and User Interface Module may provide interfaces accessible via computing devices and enable healthcare administrators to interact with the patient risk management platform. A Database Engine may manage structured data storage and retrieval operations for the Data Repository.

Conventional healthcare management platforms often process patient risk assessments in a reactive manner, with limited ability to correlate individual patient vulnerabilities with forecasted environmental conditions or automate proactive patient outreach based on integrated risk assessments. The disclosed embodiments address this problem by combining patient health data integration with environmental forecasting, medication effectiveness analysis, and automated AI-driven patient outreach to actively prevent hospitalizations before adverse environmental events occur. This configuration enables automated, data-driven risk prediction that can dynamically trigger proactive patient communication based on patient-specific vulnerabilities to forecasted environmental conditions while maintaining complete interaction records in electronic health record systems.

In practice and in use, the system may be deployed by a healthcare organization such as a health insurance payor, healthcare provider network, or care management organization that serves patient populations vulnerable to adverse environmental conditions. When the Environmental Data Module receives forecasted environmental condition data indicating that environmental thresholds will be exceeded during a future time period, the Data Integration Module may extract patient-level health data including medical histories, current medication regimens, and chronic condition indicators from connected healthcare data systems. The Risk Prediction Engine may correlate the patient-level health data with the forecasted environmental condition data to generate patient-specific hospitalization risk scores, including identifying patients whose current medication regimens may experience reduced effectiveness under the forecasted environmental conditions. The Orchestration Engine may compare risk scores to predefined thresholds and generate outreach target lists based on patient contact permissions. The Automated Outreach Module may deploy voice artificial intelligence agents and text artificial intelligence agents to deliver personalized health guidance to patients on the target lists prior to the onset of the adverse environmental conditions. The Clinical Summarization Engine may generate interaction summaries and store them in electronic health record systems to document the proactive outreach activities.

In this way, the system may improve patient health outcomes by automating the identification of at-risk patients and enabling proactive outreach before adverse environmental events occur, reducing reliance on reactive intervention after patients experience health deterioration, and preventing hospitalizations through timely delivery of personalized health guidance. The forecasted environmental data integration enables proactive intervention timing rather than reactive response to current conditions. The medication effectiveness analysis functionality identifies patients whose specific medication regimens create heightened vulnerability to forecasted environmental conditions. The dual-channel AI outreach capability enables scalable patient communication that would be impractical using human nurses alone, particularly given the temporal unpredictability of environmental events. The Clinical Summarization Engine preserves detailed interaction records, as such, the system provides documentation of proactive outreach activities for care coordination and compliance purposes. These capabilities can reduce hospitalization rates by enabling preventive intervention, improve resource utilization by automating outreach that would otherwise require manual nurse effort, and optimize intervention timing by correlating patient vulnerabilities with forecasted environmental conditions.

Various implementations of the present disclosure involve the technical field of computer-based healthcare risk prediction and patient outreach systems for preventing hospitalization due to adverse environmental conditions, including executing algorithms to extract and correlate patient health data with environmental forecasts, calculating patient-specific hospitalization risk scores based on medication effectiveness under environmental conditions, generating outreach target lists based on risk thresholds and contact permissions, deploying artificial intelligence agents to conduct patient communications, and synchronizing interaction summaries with electronic health record systems. These operations are inherently computer-based and cannot be performed in the human mind or using pen and paper due to the volume, speed, and complexity of the data being processed. For example, the system executes the steps of receiving forecasted environmental data from network-connected environmental data sources, extracting patient health data from multiple healthcare data systems, correlating patient medication regimens with environmental condition impacts based on clinical correlation data, calculating risk scores for potentially thousands of patients simultaneously, generating differentiated outreach target lists based on risk severity, and deploying AI agents to conduct patient communications at calculated outreach initiation times preceding forecasted environmental threshold exceedances. The present disclosure amounts to more than merely implementing a generic computer as a tool to gather, analyze, and output data because the claimed operations improve the field of healthcare risk management by enabling automated, proactive intervention that prevents hospitalizations rather than merely documenting patient conditions after adverse events occur. In particular, the speed at which the steps of the present disclosure occur to effectuate the disclosed method, system, or product would involve processing forecasted environmental data, correlating that data with patient health records across multiple data sources, calculating risk scores incorporating medication effectiveness impacts, and initiating AI-driven patient outreach at times calculated to precede forecasted environmental threshold exceedances. That is, the steps of the present method, system, or product are impossible to accomplish on pen and paper, cannot be accomplished as a method of organizing human activity, and amount to more than merely gathering, analyzing, and outputting data.

Various implementations of the present disclosure include executing computer-implemented risk prediction algorithms, environmental data processing routines, and AI agent deployment systems on computing hardware to enable proactive patient outreach in advance of adverse environmental conditions. The computing system implements these algorithms when it performs tasks such as parsing forecasted environmental values for heat index, air quality index, pollen count, and cold temperature, comparing forecasted values to predefined environmental thresholds, retrieving condition-specific susceptibility values from clinical correlation databases, determining predicted reductions in medication effectiveness based on correlations between medication types and environmental conditions, aggregating weighted chronic condition factors to calculate patient-specific hospitalization risk scores, and calculating outreach initiation times that precede forecasted environmental threshold exceedances by predefined lead intervals. In particular, the speed at which the system processes forecasted environmental data from environmental data sources, correlates that data with patient health records from multiple healthcare systems, calculates risk scores incorporating medication-environment interactions, and deploys AI agents to conduct patient communications would involve continuous, high-frequency data processing across networked systems. As such, the present disclosure would be impossible to accomplish on pen and paper or in the human mind due to the volume of patient health data being processed, the complexity of medication-environment correlation calculations, and the speed required to initiate proactive outreach before forecasted environmental conditions materialize.

In some embodiments, the Risk Prediction Engine processes risk assessment requests by coordinating data retrieval queries to the Data Integration Module and Environmental Data Module, applying correlation algorithms that evaluate patient-specific vulnerabilities to forecasted environmental conditions, and generating patient-specific hospitalization risk scores with itemized risk factor breakdowns. The Data Integration Module executes data extraction operations against medical and pharmacy claims systems, electronic health record systems, and care management systems to retrieve patient-level health data including medical histories, current medication regimens, chronic condition indicators, and care management encounter records. The Environmental Data Module receives forecasted environmental condition data from environmental data sources, parses forecasted values for heat index, air quality index, pollen count, and cold temperature, compares each forecasted value to predefined environmental thresholds, and generates environmental threshold exceedance indicators. The Orchestration Engine applies conditional logic that integrates risk scores with patient contact permissions to determine which patients should be assigned to voice outreach target lists versus text outreach target lists. The Automated Outreach Module calculates outreach initiation times and deploys voice artificial intelligence agents and text artificial intelligence agents to deliver personalized health guidance. The foregoing operations are executed as sequences of data extraction queries, algorithmic correlations, conditional logic evaluations, and network communications across distributed systems, and cannot practically be performed in the human mind or on paper. These concrete processing steps of patient health data integration, environmental forecast correlation, medication effectiveness analysis, risk score calculation, and AI-driven patient outreach provide a specific improvement to healthcare risk management systems by enabling proactive prevention of hospitalizations through automated intervention mechanisms, rather than a mere abstract idea of organizing human activity.

The disclosed patient risk management platform incorporates several technical features that distinguish it from conventional healthcare management systems. In some embodiments, the platform may implement forecasted environmental data integration that correlates predicted environmental conditions with patient-specific health vulnerabilities. Unlike conventional systems that issue broad population-level alerts based on current environmental conditions, the disclosed platform may analyze forecasted environmental condition data for future time periods and correlate those forecasts with individual patient health profiles to identify patients at heightened risk before adverse conditions materialize. The Environmental Data Module may receive forecasted values for heat index, air quality index, pollen count, and cold temperature, compare each forecasted value to predefined environmental thresholds, and generate environmental threshold exceedance indicators that trigger downstream risk assessment and outreach processes.

In some embodiments, the platform may implement medication effectiveness correlation that identifies patients whose current medication regimens may experience reduced effectiveness under forecasted environmental conditions. Unlike conventional systems that assess patient risk based on diagnosis codes and chronic conditions alone, the disclosed platform may retrieve condition-specific susceptibility values from a clinical correlation database, wherein the condition-specific susceptibility values are derived from historical hospitalization data correlating chronic conditions with hospitalization occurrences during prior environmental events. The Risk Prediction Engine may determine predicted reductions in medication effectiveness for specific medication types under specific environmental conditions, such as reduced bronchodilator effectiveness during high heat index conditions, increased adverse reaction risk for patients on antidepressant medications during high heat index conditions, or reduced inhaler effectiveness during high air quality index conditions. This medication-environment correlation may enable identification of patients whose specific treatment regimens create heightened vulnerability to forecasted environmental conditions.

In some embodiments, the platform may implement weighted chronic condition aggregation that calculates patient-specific hospitalization risk scores by assigning weights to each chronic condition based on condition-specific susceptibility values and aggregating the assigned weights. The Risk Prediction Engine may identify a plurality of chronic conditions from the patient-level health data, retrieve condition-specific susceptibility values from the clinical correlation database for each identified chronic condition, assign a weight to each chronic condition based on the retrieved susceptibility value, and calculate the patient-specific hospitalization risk score by aggregating the weights assigned to each chronic condition present in the patient-level health data. The Risk Prediction Engine may further generate a risk factor breakdown comprising an itemized list identifying each contributing factor to the patient-specific hospitalization risk score and a corresponding weight assigned to each contributing factor. This itemized risk factor breakdown may enable the AI agents to deliver personalized health guidance that addresses each patient's specific risk factors.

In some embodiments, the platform may implement dual-channel AI outreach that deploys both voice artificial intelligence agents and text artificial intelligence agents to conduct patient communications based on risk severity. The Orchestration Engine may determine whether each patient's hospitalization risk score exceeds a high-risk threshold and may assign patients exceeding the high-risk threshold to a voice outreach target list while assigning patients below the high-risk threshold to a text outreach target list. The voice artificial intelligence agent may be configured to conduct a spoken dialogue with the patient to deliver the personalized health guidance and to receive a verbal response from the patient. The text artificial intelligence agent may be configured to conduct a text-based messaging exchange with the patient to deliver the personalized health guidance and to receive a text response from the patient. This differentiated channel assignment may enable more intensive voice-based interaction for highest-risk patients while enabling scalable text-based outreach for moderate-risk patients.

In some embodiments, the platform may implement proactive outreach timing that calculates outreach initiation times preceding forecasted environmental threshold exceedances. The Automated Outreach Module may determine an onset time at which the forecasted environmental condition data is predicted to exceed the predefined environmental threshold, calculate an outreach initiation time that precedes the onset time by a predefined lead interval, and initiate the proactive patient communication at the outreach initiation time such that each patient on the voice outreach target list and each patient on the text outreach target list receives the personalized health guidance prior to the onset time. This proactive timing capability may enable patients to receive health guidance and take preventive actions before adverse environmental conditions materialize, rather than receiving reactive communications after conditions have already deteriorated.

In some embodiments, the platform may implement personalized health guidance delivery that provides patients with information tailored to their specific risk factors and environmental vulnerabilities. The personalized health guidance delivered by the AI agents may comprise educational information regarding the forecasted environmental condition data, recommended preventive actions based on the current medication regimen, or locations of resource facilities such as cooling centers during heat events. The AI agents may reference the risk factor breakdown generated by the Risk Prediction Engine to address each patient's specific chronic conditions, medication considerations, and environmental vulnerabilities during the outreach communication.

In some embodiments, the platform may implement clinical summarization and electronic health record integration that documents proactive outreach activities and stores interaction summaries in patient health records. The Clinical Summarization Engine may receive interaction data from the Automated Outreach Module, generate an interaction summary documenting the proactive patient communication, record the timestamp of the communication, record the communication channel used, record the response received from the patient, and generate recommended follow-up actions based on the patient's response. The Clinical Summarization Engine may store the interaction summary in the Data Repository and transmit the interaction summary to the electronic health record system for storage. This documentation capability may enable care teams to review proactive outreach activities, coordinate follow-up care based on patient responses, and maintain comprehensive records of preventive intervention efforts.

In some embodiments, the platform may implement patient contact permission enforcement that applies regulatory and preference-based rules to outreach activities. The Orchestration Engine may retrieve patient contact permissions from the Data Repository, wherein the patient contact permissions comprise permitted contact time windows, preferred communication channels, contact frequency limitations, or do-not-contact indicators. The Orchestration Engine may apply the patient contact permissions when generating outreach target lists to ensure that proactive communications comply with patient preferences and regulatory requirements governing healthcare communications.

The integration with multiple healthcare data sources may represent a departure from conventional patient risk systems that analyze data from single sources in isolation. In some embodiments, the Data Integration Module may interface with medical and pharmacy claims systems, electronic health record systems, and care management systems via application programming interfaces or custom data extraction routines to compile comprehensive patient-level health data. The Data Integration Module may extract medical history and diagnosis codes from medical claims systems, extract current medication regimens and prescription fill history from pharmacy claims systems, extract chronic condition indicators and comorbidity data from electronic health record systems, and extract care management encounter records from care management systems. By aggregating patient data from multiple sources, the platform may generate more comprehensive patient health profiles than systems limited to single data sources, enabling more accurate risk prediction based on complete views of patient health status, medication regimens, and care history.

The embedded medication effectiveness analysis functionality ensures that medication-environment interactions are evaluated as part of risk assessment rather than being overlooked by systems that assess diagnosis codes and environmental conditions independently. In some embodiments, the Risk Prediction Engine may retrieve condition-specific susceptibility values from the clinical correlation database that quantify correlations between specific chronic conditions, specific medication types, and hospitalization occurrences during prior environmental events. The system may identify patients whose medication effectiveness may be compromised by forecasted environmental conditions, such as patients on medications that affect thermoregulation during heat events or patients on respiratory medications during air quality events. This embedded analysis may address a common limitation in conventional systems where medication-environment interactions are not systematically evaluated during patient risk assessment.

These technical features may collectively transform healthcare risk management from a reactive documentation process into a proactive prevention system. Whereas conventional systems may identify at-risk patients after adverse events occur and rely on manual nurse outreach that cannot scale rapidly during unpredictable environmental events, the disclosed platform may identify at-risk patients before forecasted environmental conditions materialize and may deploy AI agents capable of conducting thousands of patient communications simultaneously. By integrating patient health data extraction, environmental forecast correlation, medication effectiveness analysis, risk score calculation, dual-channel AI outreach, and clinical summarization into a unified prevention platform with proactive timing capabilities, the system may address fundamental limitations in current healthcare risk management approaches that separate risk identification from intervention delivery and rely on human resources that cannot scale to meet demand during time-sensitive environmental events.

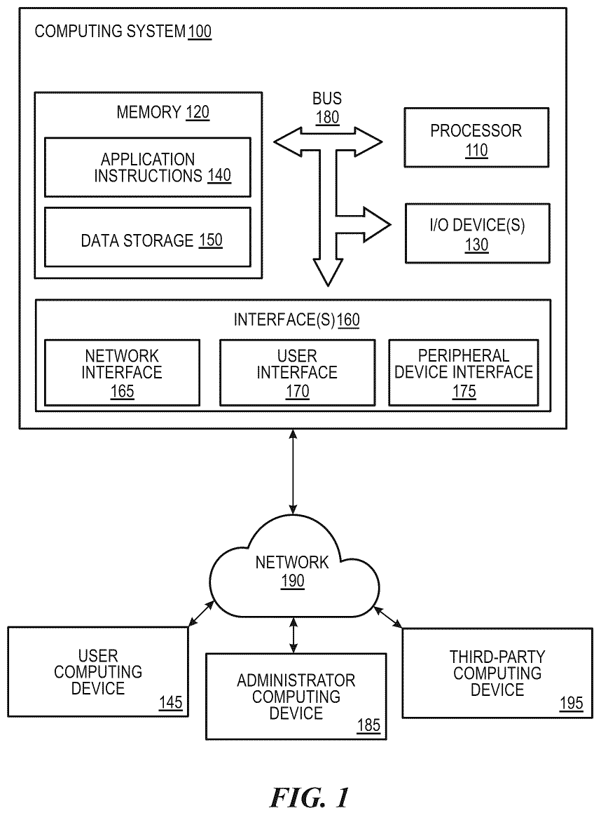

FIG. 1 illustrates an example of a computing system 100 that may provide the execution environment for implementing the processes and methods described herein. The computing system 100 may take various forms depending on deployment context, including but not limited to: a desktop or laptop computer, a tablet or smartphone, a server in a data center, a network appliance, a mainframe computer, a workstation, or a cloud-hosted virtual machine. In some embodiments, the computing system 100 may correspond to a distributed computing environment, such as a cluster of servers executing containerized workloads (e.g., Docker, Kubernetes), or an edge device integrated into Internet of Things (IoT) environments. In other embodiments, the computing system 100 may be embedded in another device, such as a vehicle infotainment unit, a medical diagnostic machine, an industrial robot controller, or a wearable computing device.

The computing system 100 includes one or more processors 110 operably coupled to a memory 120 via a system bus 180. The processor 110 may be implemented as a general-purpose central processing unit (CPU), a graphics processing unit (GPU), a tensor processing unit (TPU), a digital signal processor (DSP), or any combination thereof. In some embodiments, the processor 110 may be an application-specific integrated circuit (ASIC) optimized for a particular workload, a field-programmable gate array (FPGA), or a quantum or neuromorphic processor in advanced implementations. The processor 110 may include single-core, multi-core, or many-core configurations and may support hardware virtualization, multithreading, or parallel execution environments to optimize system performance.

The memory 120 may include volatile memory, nonvolatile memory, or a combination thereof. Volatile memory may include system RAM, cache memory, or high-bandwidth memory (HBM). Nonvolatile memory may include flash storage, solid-state drives (SSD), magnetic hard disk drives (HDD), optical storage devices, or persistent memory technologies such as Intel Optane. The memory 120 stores application instructions 140 for carrying out the functionalities described herein and data storage 150 for maintaining information related to system operations. The application instructions 140 may include code written in languages such as C, C++, Java, Python, Go, Rust, or JavaScript, as well as machine learning models trained using frameworks such as TensorFlow or PyTorch. The data storage 150 may contain structured information such as relational database records, unstructured data such as text or images, or real-time telemetry streams. In cloud-based embodiments, the memory 120 may represent scalable storage resources provisioned on-demand through Infrastructure-as-a-Service (IaaS) providers.

The computing system 100 may also include one or more input/output (I/O) devices 130. These devices may encompass visual output devices such as monitors, head-mounted displays, augmented reality (AR) glasses, or projectors; input devices such as keyboards, mice, touchscreens, styluses, or game controllers; and sensor devices such as microphones, cameras, depth sensors, biometric scanners, or environmental sensors. In industrial or medical environments, the I/O devices 130 may include robotic actuators, infusion pumps, or diagnostic imaging scanners. In vehicular environments, the I/O devices 130 may include in-cabin displays, steering sensors, and connected infotainment systems.

The computing system 100 further comprises one or more interfaces 160 that enable communication with other systems, users, or peripheral components. The network interface 165 allows the computing system 100 to exchange data with external systems across a network 190 using wired or wireless protocols. Example communication standards include Ethernet, Wi-Fi, Bluetooth, 5G, Long-Term Evolution (LTE), satellite communication, or emerging protocols such as Wi-Fi 7 or ultra-wideband (UWB). In some embodiments, the network interface 165 supports secure protocols such as HTTPS, TLS, or VPN tunneling to ensure authenticated and encrypted data transfer. The user interface 170 may include APIs, graphical user interfaces (GUIs), command-line interfaces (CLIs), or natural language interfaces enabled through speech recognition or chatbot systems. The peripheral device interface 175 enables connectivity with external hardware such as printers, external storage arrays, or specialized scientific equipment.

The network 190 represents any communication infrastructure capable of facilitating data exchange between computing entities. In some embodiments, the network 190 corresponds to a local area network (LAN) within a home or enterprise environment. In other embodiments, the network 190 may be a wide area network (WAN), a metropolitan area network (MAN), a peer-to-peer (P2P) communication mesh, or the global Internet. The network 190 may employ cloud orchestration layers, software-defined networking (SDN), or edge computing gateways. In high-security applications, the network 190 may implement firewalls, intrusion detection systems, or zero-trust architectures to protect transmitted data.

The computing system 100 is illustrated as being in communication with multiple external devices, including a user computing device 145, an administrator computing device 185, and a third-party computing device 195. The user computing device 145 may be a smartphone, tablet, laptop, or smart appliance configured to execute client-side applications or interact with system services. The administrator computing device 185 may be a workstation or remote management console configured to perform oversight functions such as monitoring, auditing, updating, or troubleshooting. The third-party computing device 195 may represent a partner system, vendor service, or external application interface that exchanges data with the computing system 100 via secure APIs. In cloud or SaaS embodiments, these devices may also include external microservices, data warehouses, or federated learning nodes.

In some embodiments, the computing system 100 may be deployed in a client-server model, where the computing system 100 acts as a backend server managing requests from client devices. In other embodiments, the computing system 100 may function within a cloud-native environment, operating as a microservice within a container orchestration platform. In edge deployments, the computing system 100 may be optimized for low-latency local processing, while synchronizing with centralized cloud infrastructure for data persistence and global coordination.

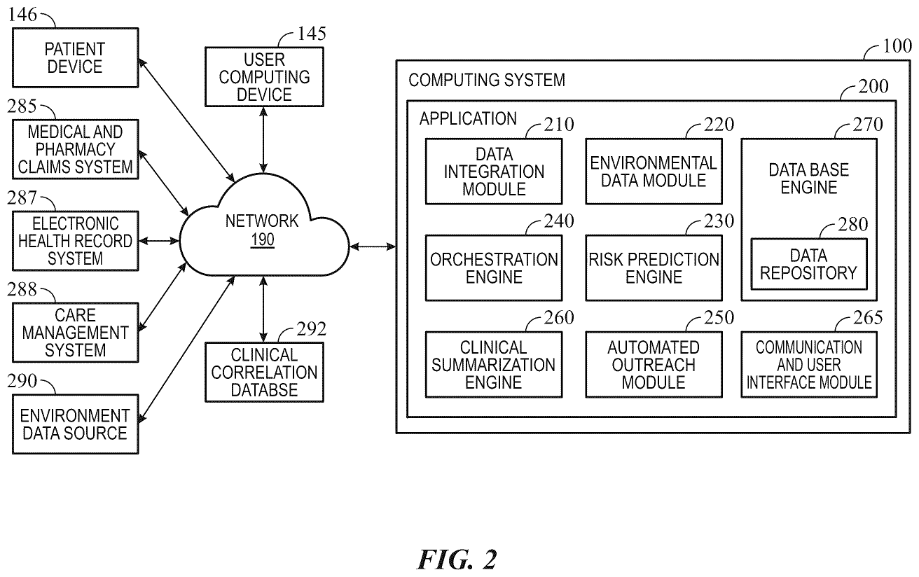

FIG. 2 illustrates an example computer architecture for the application program 200 operated via the computing system 100. The computer system 100 comprises several modules and engines configured to execute the functionalities of the application program 200, and a database engine 270 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a Data Integration Module 210, an Environmental Data Module 220, a Risk Prediction Engine 230, an Orchestration Engine 240, an Automated Outreach Module 250, a Clinical Summarization Engine 260, and a Communication and User Interface Module 265. The application program 200 further interfaces with a Database Engine 270 that manages operations for the Data Repository 280. The computing system 100 communicates via network 190 with external systems including user computing device 145, patient device 146, medical and pharmacy claims system 285, electronic health record system 287, care management system 288, environmental data source 290, and clinical correlation database 292.

In some embodiments, the Data Integration Module 210 may be configured to extract patient-level health data from multiple healthcare data systems and compile the extracted data into a unified patient health profile for use by the Risk Prediction Engine 230. The Data Integration Module 210 may establish connections with the medical and pharmacy claims system 285, electronic health record system 287, and care management system 288 via the network 190 using application programming interfaces (APIs) or custom data extraction routines. In some embodiments, the Data Integration Module 210 may receive a patient data extraction request from the Risk Prediction Engine 230 containing patient identifiers for patients requiring risk assessment. The Data Integration Module 210 may execute data extraction operations against each connected healthcare data system to retrieve patient-level health data associated with the specified patient identifiers.

In some embodiments, the Data Integration Module 210 may extract medical history and diagnosis codes from the medical and pharmacy claims system 285, wherein the medical history comprises records of prior healthcare encounters, procedures, and diagnosed conditions. The Data Integration Module 210 may further extract a current medication regimen and prescription fill history from the medical and pharmacy claims system 285, wherein the current medication regimen comprises a list of medications actively prescribed to the patient and the prescription fill history comprises records of when prescriptions were dispensed. The Data Integration Module 210 may extract chronic condition indicators and comorbidity data from the electronic health record system 287, wherein the chronic condition indicators identify persistent health conditions such as asthma, chronic obstructive pulmonary disease, cardiovascular disease, or diabetes that may increase patient vulnerability to environmental conditions. The Data Integration Module 210 may extract care management encounter records from the care management system 288, wherein the care management encounter records comprise documentation of prior interactions between care managers and the patient.

In some embodiments, the Data Integration Module 210 may compile the extracted data into patient-level health data comprising at least one of a medical history, a current medication regimen, or a chronic condition indicator. The compiled patient-level health data may further comprise at least one of a diagnosis code, a procedure code, a prescription fill history, a comorbidity indicator, or a care management encounter record. The Data Integration Module 210 may transmit the compiled patient-level health data to the Risk Prediction Engine 230 for correlation with forecasted environmental condition data. The Data Integration Module 210 may store extracted patient data in the Data Repository 280 via the Database Engine 270 to enable subsequent retrieval without requiring repeated extraction from external systems.

In some embodiments, the Environmental Data Module 220 may be configured to receive forecasted environmental condition data from the environmental data source 290 and determine when forecasted values exceed predefined environmental thresholds. The Environmental Data Module 220 may establish a connection with the environmental data source 290 via the network 190 using an API to retrieve forecasted environmental condition data for geographic regions associated with patient populations served by the healthcare organization. In some embodiments, the environmental data source 290 may comprise at least one of a governmental weather data source, a governmental air quality monitoring data source, or a third-party environmental data vendor. The Environmental Data Module 220 may receive forecasted environmental condition data comprising predicted values for at least one of a heat index, an air quality index, a pollen count, or a cold temperature for a future time period.

In some embodiments, the Environmental Data Module 220 may parse the forecasted environmental condition data to extract individual forecasted values for each environmental parameter. The Environmental Data Module 220 may retrieve predefined environmental thresholds from the Data Repository 280 via the Database Engine 270, wherein each predefined environmental threshold specifies a value above which the corresponding environmental parameter is considered to pose a risk to patient health. The Environmental Data Module 220 may compare each forecasted value to its corresponding predefined environmental threshold by executing numerical comparison operations that evaluate whether the forecasted value exceeds the threshold value. In some embodiments, the Environmental Data Module 220 may determine that the forecasted environmental condition data exceeds a predefined environmental threshold when any forecasted value for heat index, air quality index, pollen count, or cold temperature exceeds its corresponding threshold.

In some embodiments, the Environmental Data Module 220 may generate an environmental threshold exceedance indicator when any forecasted value exceeds its corresponding predefined environmental threshold. The environmental threshold exceedance indicator may identify which specific environmental parameters are forecasted to exceed thresholds, the magnitude by which forecasted values exceed thresholds, and the future time period during which threshold exceedances are predicted to occur. The Environmental Data Module 220 may transmit the forecasted environmental condition data and the environmental threshold exceedance indicator to the Risk Prediction Engine 230 for correlation with patient-level health data. The Environmental Data Module 220 may continuously monitor the environmental data source 290 and transmit updated forecasted environmental condition data to the Risk Prediction Engine 230 when the forecasted environmental condition data changes.

In some embodiments, the Risk Prediction Engine 230 may be configured to generate patient-specific hospitalization risk scores and risk factor breakdowns by correlating patient-level health data with forecasted environmental condition data. The Risk Prediction Engine 230 may receive patient-level health data from the Data Integration Module 210 and forecasted environmental condition data from the Environmental Data Module 220. In some embodiments, the Risk Prediction Engine 230 may initiate patient data extraction and environmental data retrieval by transmitting requests to the Data Integration Module 210 and Environmental Data Module 220 when the Environmental Data Module 220 detects that forecasted environmental condition data exceeds predefined environmental thresholds.

In some embodiments, the Risk Prediction Engine 230 may identify the current medication regimen from the patient-level health data and retrieve condition-specific susceptibility values from the clinical correlation database 292 via the network 190. The clinical correlation database 292 may store condition-specific susceptibility values derived from historical hospitalization data correlating chronic conditions with hospitalization occurrences during prior environmental events. Each condition-specific susceptibility value may quantify a correlation between a specific chronic condition and an increased hospitalization likelihood when exposed to specific forecasted environmental condition data. In some embodiments, the condition-specific susceptibility values may be calculated by analyzing historical records of patient hospitalizations that occurred during prior environmental events and identifying statistical correlations between specific chronic conditions, specific medication types, and hospitalization occurrences.

In some embodiments, the Risk Prediction Engine 230 may determine a predicted reduction in medication effectiveness for the current medication regimen when the forecasted environmental condition data exceeds the predefined environmental threshold during the future time period. The predicted reduction in medication effectiveness may comprise at least one of a reduced bronchodilator effectiveness during a high heat index condition, an increased adverse reaction risk for a patient on an antidepressant medication during a high heat index condition, or a reduced inhaler effectiveness during a high air quality index condition. The Risk Prediction Engine 230 may identify relationships between specific medication types and specific environmental conditions by querying the clinical correlation database 292 for medication-environment interaction data that documents how environmental conditions affect medication performance.

In some embodiments, the Risk Prediction Engine 230 may assign a weight to each chronic condition based on the condition-specific susceptibility value retrieved from the clinical correlation database 292. The Risk Prediction Engine 230 may identify a plurality of chronic conditions from the patient-level health data by parsing diagnosis codes and chronic condition indicators extracted by the Data Integration Module 210. For each chronic condition identified in the patient-level health data, the Risk Prediction Engine 230 may retrieve the corresponding condition-specific susceptibility value and assign a weight corresponding to the retrieved susceptibility value. The Risk Prediction Engine 230 may calculate the patient-specific hospitalization risk score by aggregating the weights assigned to each of the plurality of chronic conditions present in the patient-level health data.

In some embodiments, the Risk Prediction Engine 230 may generate a risk factor breakdown comprising an itemized list identifying each contributing factor to the patient-specific hospitalization risk score and a corresponding weight assigned to each contributing factor. The risk factor breakdown may identify specific chronic conditions, specific medications, and specific environmental vulnerabilities that contribute to the patient-specific hospitalization risk score. The Risk Prediction Engine 230 may transmit the patient-specific hospitalization risk score and the risk factor breakdown to the Orchestration Engine 240 for generation of outreach target lists. The Risk Prediction Engine 230 may store calculated risk scores and risk factor breakdowns in the Data Repository 280 via the Database Engine 270 to enable historical tracking and analysis.

In some embodiments, the Orchestration Engine 240 may be configured to generate a voice outreach target list and a text outreach target list based on patient-specific hospitalization risk scores and patient contact permissions. The Orchestration Engine 240 may receive patient-specific hospitalization risk scores and risk factor breakdowns from the Risk Prediction Engine 230. In some embodiments, the Orchestration Engine 240 may compare each patient-specific hospitalization risk score to a predefined risk threshold by executing numerical comparison operations that evaluate whether the risk score exceeds the threshold value. The predefined risk threshold may be configured by healthcare administrators via the Communication and User Interface Module 265 and stored in the Data Repository 280.

In some embodiments, the Orchestration Engine 240 may retrieve patient contact permissions from the Data Repository 280 via the Database Engine 270. The patient contact permissions may define permitted timing and permitted communication channels for contacting each patient. In some embodiments, the patient contact permissions may comprise at least one of a permitted contact time window specifying hours during which the patient may be contacted, a preferred communication channel indicating whether the patient prefers voice calls or text messages, a contact frequency limitation specifying maximum contact attempts within a time period, or a do-not-contact indicator specifying that the patient should not receive automated outreach. The Orchestration Engine 240 may apply the patient contact permissions when generating outreach target lists to ensure that proactive communications comply with patient preferences and regulatory requirements.

In some embodiments, the Orchestration Engine 240 may determine whether each patient-specific hospitalization risk score exceeds a high-risk threshold. The high-risk threshold may be configured to differentiate between patients requiring more intensive voice-based outreach and patients suitable for text-based outreach. The Orchestration Engine 240 may assign each patient to at least one of the voice outreach target list or the text outreach target list based on a severity level of the patient-specific hospitalization risk score. In some embodiments, patients having a patient-specific hospitalization risk score exceeding the high-risk threshold may be assigned to the voice outreach target list, and patients having a patient-specific hospitalization risk score below the high-risk threshold may be assigned to the text outreach target list. The Orchestration Engine 240 may transmit the voice outreach target list, the text outreach target list, and the risk factor breakdown to the Automated Outreach Module 250.

In some embodiments, the Automated Outreach Module 250 may be configured to initiate proactive patient communication to each patient on the voice outreach target list via a voice artificial intelligence agent and to each patient on the text outreach target list via a text artificial intelligence agent. The Automated Outreach Module 250 may receive the voice outreach target list, the text outreach target list, and the risk factor breakdown from the Orchestration Engine 240. In some embodiments, the Automated Outreach Module 250 may determine an onset time at which the forecasted environmental condition data is predicted to exceed the predefined environmental threshold. The onset time may be extracted from the environmental threshold exceedance indicator generated by the Environmental Data Module 220.

In some embodiments, the Automated Outreach Module 250 may calculate an outreach initiation time that precedes the onset time by a predefined lead interval. The predefined lead interval may be configured by healthcare administrators to ensure that patients receive health guidance with sufficient time to take preventive actions before adverse environmental conditions materialize. The Automated Outreach Module 250 may initiate the proactive patient communication at the outreach initiation time such that each patient on the voice outreach target list and each patient on the text outreach target list receives personalized health guidance prior to the onset time.

In some embodiments, the voice artificial intelligence agent may be configured to conduct a spoken dialogue with the patient to deliver the personalized health guidance and to receive a verbal response from the patient. The voice artificial intelligence agent may initiate outbound telephone calls to patient devices 146 associated with patients on the voice outreach target list. The voice artificial intelligence agent may use speech synthesis to deliver personalized health guidance based on the risk factor breakdown and may use speech recognition to receive and interpret verbal responses from the patient. In some embodiments, the text artificial intelligence agent may be configured to conduct a text-based messaging exchange with the patient to deliver the personalized health guidance and to receive a text response from the patient. The text artificial intelligence agent may transmit text messages to patient devices 146 associated with patients on the text outreach target list and may receive text message responses from patients.

In some embodiments, the personalized health guidance delivered by the voice artificial intelligence agent and the text artificial intelligence agent may comprise at least one of educational information regarding the forecasted environmental condition data, a recommended preventive action based on the current medication regimen, or a location of a resource facility. The resource facility may comprise a cooling center during heat events, an air-conditioned public facility during air quality events, or other community resources appropriate to the forecasted environmental condition. The Automated Outreach Module 250 may reference the risk factor breakdown when generating personalized health guidance to address each patient's specific chronic conditions, medication considerations, and environmental vulnerabilities. The Automated Outreach Module 250 may transmit interaction data to the Clinical Summarization Engine 260, wherein the interaction data comprises records of communications conducted with patients and responses received.

In some embodiments, the Clinical Summarization Engine 260 may be configured to generate an interaction summary documenting the proactive patient communication and to store the interaction summary in the electronic health record system 287. The Clinical Summarization Engine 260 may receive interaction data from the Automated Outreach Module 250 comprising records of voice and text communications conducted with patients. In some embodiments, the Clinical Summarization Engine 260 may generate the interaction summary by compiling information from the interaction data into a structured documentation format suitable for storage in electronic health records.

In some embodiments, the interaction summary may comprise at least one of a timestamp of the proactive patient communication, a communication channel used indicating whether the communication was conducted via voice call or text message, a response received from the patient, or a recommended follow-up action. The Clinical Summarization Engine 260 may record the timestamp of the proactive patient communication by extracting date and time information from the interaction data. The Clinical Summarization Engine 260 may record the communication channel used by identifying whether the communication was conducted by the voice artificial intelligence agent or the text artificial intelligence agent. The Clinical Summarization Engine 260 may record the response received from the patient by extracting patient response content from the interaction data. In some embodiments, the Clinical Summarization Engine 260 may generate a recommended follow-up action based on the patient response, wherein the recommended follow-up action may comprise scheduling a care management call, arranging transportation to a resource facility, or escalating to a human care manager for patients expressing distress or confusion.

In some embodiments, the Clinical Summarization Engine 260 may store the interaction summary in the Data Repository 280 via the Database Engine 270. The Clinical Summarization Engine 260 may transmit the interaction summary to the electronic health record system 287 via the network 190 for storage in the patient's health record. The transmission may be accomplished using healthcare interoperability standards such as Health Level Seven (HL7) or Fast Healthcare Interoperability Resources (FHIR) to ensure compatibility with electronic health record systems. The stored interaction summaries may enable care teams to review proactive outreach activities, coordinate follow-up care based on patient responses, and maintain comprehensive records of preventive intervention efforts.

In some embodiments, the Communication and User Interface Module 265 may be configured to facilitate interactions between healthcare administrators and the patient risk management platform. The Communication and User Interface Module 265 may generate user interfaces that are transmitted to user computing devices 145 via the network 190 for display on device screens. In some embodiments, the generated interfaces may enable healthcare administrators to configure predefined environmental thresholds, predefined risk thresholds, and high-risk thresholds used by the Environmental Data Module 220, Risk Prediction Engine 230, and Orchestration Engine 240. The Communication and User Interface Module 265 may enable healthcare administrators to view patient-specific hospitalization risk scores, review risk factor breakdowns, and monitor outreach activities across patient populations.

In some embodiments, the Communication and User Interface Module 265 may enable healthcare administrators to configure patient contact permissions and predefined lead intervals used by the Orchestration Engine 240 and Automated Outreach Module 250. The Communication and User Interface Module 265 may implement authentication mechanisms to verify user identity before granting access to the patient risk management platform and may enforce access controls that restrict which functions and data each user may access based on assigned permissions. In some embodiments, the Communication and User Interface Module 265 may support access from mobile devices such as smartphones and tablets, enabling healthcare administrators to monitor and configure the patient risk management platform from remote locations.

In some embodiments, the Database Engine 270 may be configured to manage data storage and retrieval operations for the Data Repository 280. The Database Engine 270 may execute database queries submitted by other components of the application program 200, maintain indexes that enable efficient data retrieval, and ensure data integrity by enforcing constraints on stored data. In some embodiments, the Database Engine 270 may manage storage of patient-level health data, forecasted environmental condition data, patient-specific hospitalization risk scores, risk factor breakdowns, patient contact permissions, outreach target lists, and interaction summaries in the Data Repository 280. The Database Engine 270 may support concurrent access by multiple components, enabling the Data Integration Module 210, Environmental Data Module 220, Risk Prediction Engine 230, Orchestration Engine 240, Automated Outreach Module 250, and Clinical Summarization Engine 260 to read and write data simultaneously without conflicts.

In some embodiments, the Data Repository 280 may store structured data used by the application program 200. The Data Repository 280 may contain patient-level health data including medical histories, current medication regimens, chronic condition indicators, and care management encounter records extracted by the Data Integration Module 210. The Data Repository 280 may contain forecasted environmental condition data including heat index values, air quality index values, pollen count values, and cold temperature values received by the Environmental Data Module 220. The Data Repository 280 may contain patient-specific hospitalization risk scores, risk factor breakdowns, and condition-specific susceptibility values generated or retrieved by the Risk Prediction Engine 230. The Data Repository 280 may contain patient contact permissions, outreach target lists, and predefined thresholds used by the Orchestration Engine 240. The Data Repository 280 may contain interaction summaries generated by the Clinical Summarization Engine 260. In some embodiments, the Data Repository 280 may be implemented as a relational database, a NoSQL database, or a distributed storage system depending on system requirements and data volumes.

In some embodiments, the computing system 100 may be implemented as a cloud-based server configured to synchronize the patient-level health data and the forecasted environmental condition data across a plurality of healthcare facilities. The cloud-based architecture may enable healthcare organizations operating multiple facilities or serving geographically distributed patient populations to maintain centralized risk prediction and outreach coordination. In some embodiments, the network 190 may be a public or private data network, such as the Internet or a corporate intranet, enabling communication between the computing system 100, user computing device 145, patient device 146, medical and pharmacy claims system 285, electronic health record system 287, care management system 288, environmental data source 290, and clinical correlation database 292. The network 190 may utilize standard communication protocols such as Transmission Control Protocol/Internet Protocol (TCP/IP), Hypertext Transfer Protocol Secure (HTTPS), or other network protocols to facilitate data transmission between connected systems.

User computing device 145 may be a mobile device such as a smartphone or tablet, a portable computing device such as a laptop, or a fixed workstation such as a desktop computer used by healthcare administrators to access the patient risk management platform. In some embodiments, user computing device 145 may execute web browsers or dedicated applications that interface with the Communication and User Interface Module 265 to display configuration interfaces, risk scores, and outreach activity reports. Patient device 146 may be a mobile device such as a smartphone or a landline telephone used by patients to receive proactive communications from the voice artificial intelligence agent or text artificial intelligence agent. The patient device 146 may receive inbound voice calls or text messages initiated by the Automated Outreach Module 250 and may transmit patient responses back to the Automated Outreach Module 250 via the network 190.

Medical and pharmacy claims system 285 may be an external data system maintained by a healthcare payor or pharmacy benefit manager that stores medical claims records and pharmacy claims records for patient populations. In some embodiments, the medical and pharmacy claims system 285 may store diagnosis codes, procedure codes, claim dates, provider identifiers, medication identifiers, prescription fill dates, and dispensing quantities. The Data Integration Module 210 may query the medical and pharmacy claims system 285 via the network 190 using APIs or database connections to retrieve medical history, diagnosis codes, current medication regimens, and prescription fill history for patients requiring risk assessment.

Electronic health record system 287 may be an external software platform that maintains comprehensive health records for patients including clinical documentation, laboratory results, vital signs, and chronic condition indicators. In some embodiments, the electronic health record system 287 may be a commercially available electronic health record platform or a proprietary system operated by a healthcare provider organization. The Data Integration Module 210 may query the electronic health record system 287 via the network 190 to retrieve chronic condition indicators and comorbidity data for patients requiring risk assessment. The Clinical Summarization Engine 260 may transmit interaction summaries to the electronic health record system 287 via the network 190 for storage in patient health records.

Care management system 288 may be an external software platform that maintains records of care management activities including care plans, care manager assignments, and care management encounter records. In some embodiments, the care management system 288 may store documentation of prior outreach attempts, patient engagement levels, and care coordination activities. The Data Integration Module 210 may query the care management system 288 via the network 190 to retrieve care management encounter records that provide context for patient engagement history and preferences.

Environmental data source 290 may be an external data system that provides forecasted environmental condition data for geographic regions. In some embodiments, the environmental data source 290 may comprise at least one of a governmental weather data source that provides forecasted heat index and cold temperature values, a governmental air quality monitoring data source that provides forecasted air quality index values, or a third-party environmental data vendor that aggregates environmental forecasts from multiple sources. The Environmental Data Module 220 may query the environmental data source 290 via the network 190 using APIs to retrieve forecasted environmental condition data comprising predicted values for heat index, air quality index, pollen count, and cold temperature for future time periods.

Clinical correlation database 292 may be a database that stores condition-specific susceptibility values derived from historical hospitalization data correlating chronic conditions with hospitalization occurrences during prior environmental events. In some embodiments, the clinical correlation database 292 may store statistical correlations between specific chronic condition types, specific medication types, specific environmental condition types, and hospitalization rates observed during prior environmental events. The clinical correlation database 292 may store medication-environment interaction data documenting how specific environmental conditions affect the effectiveness of specific medication types. The Risk Prediction Engine 230 may query the clinical correlation database 292 via the network 190 to retrieve condition-specific susceptibility values for chronic conditions identified in patient-level health data and to retrieve medication-environment interaction data for medications in patient medication regimens.

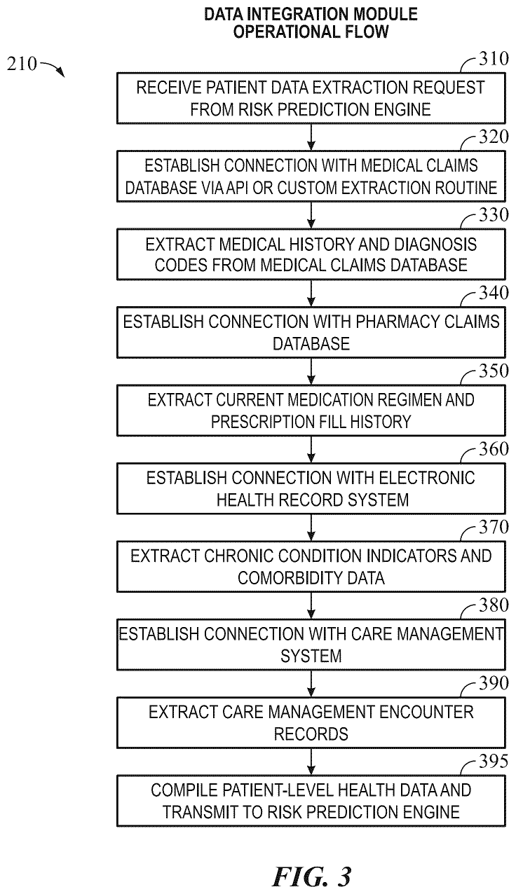

FIG. 3 is a flow diagram illustrating an exemplary operational sequence of the Data Integration Module 210 in accordance with certain embodiments. The sequence shown may be implemented as computer-executable instructions within the application program 200 operating on computing system 100 of FIG. 2, with specific operations performed by the modules depicted therein. FIG. 3 illustrates the sequential data extraction process by which the Data Integration Module 210 retrieves patient-level health data from multiple external healthcare data systems and compiles the extracted data for transmission to the Risk Prediction Engine 230.

At step 310, a patient data extraction request is received from the Risk Prediction Engine 230. This operation may be performed by the Data Integration Module 210, which monitors an inbound message queue for extraction requests. In some embodiments, the patient data extraction request may contain an array of patient identifiers, wherein each patient identifier comprises a unique alphanumeric string such as a member identifier or medical record number that uniquely identifies a patient within the healthcare organization's data systems. The request may further contain a geographic region identifier specifying the location of the forecasted environmental event, enabling the Data Integration Module 210 to identify patients residing within the affected geographic region. The Data Integration Module 210 may parse the received request by extracting the patient identifier array and storing the extracted identifiers in a local memory buffer for use in subsequent extraction steps. In some embodiments, the Data Integration Module 210 may validate the received request by verifying that required fields are present and that patient identifiers conform to expected format patterns before proceeding with extraction operations.

At step 320, a connection is established with the medical claims database via an application programming interface (API) or custom extraction routine. This operation is performed by the Data Integration Module 210, which initiates a secure network connection to the medical and pharmacy claims system 285 via the network 190. In some embodiments, the Data Integration Module 210 may establish the connection using Transport Layer Security (TLS) encryption to protect data in transit. The Data Integration Module 210 may authenticate with the medical and pharmacy claims system 285 by transmitting authentication credentials to an authentication endpoint and receiving an access token in response. The access token may contain encoded permissions specifying the data access rights granted to the Data Integration Module 210 and an expiration timestamp. In some embodiments, when the medical and pharmacy claims system 285 does not support API access, the Data Integration Module 210 may establish a connection using a custom extraction routine that opens a direct database connection and executes queries against the underlying data store.

At step 330, medical history and diagnosis codes are extracted from the medical claims database. This operation is performed by the Data Integration Module 210, which constructs and transmits data retrieval requests to the medical and pharmacy claims system 285. In some embodiments, the Data Integration Module 210 may construct a query specifying the patient identifiers extracted in step 310 and a date range parameter specifying the lookback period for claims retrieval, such as twenty-four months from the current date. The medical and pharmacy claims system 285 may process the received request by querying its claims data store for records matching the specified patient identifiers and date range. The returned claim records may contain a claim identifier, a service date, a claim type indicator distinguishing inpatient, outpatient, and emergency claims, and an array of diagnosis codes. The diagnosis codes may be formatted according to the International Classification of Diseases (ICD) coding system, wherein each diagnosis code identifies a specific diagnosed condition. The Data Integration Module 210 may parse the returned claim records by iterating through the response, extracting the diagnosis codes from each claim record, and aggregating the extracted diagnosis codes into a deduplicated set of unique diagnosis codes for each patient.

At step 340, a connection is established with the pharmacy claims database. This operation is performed by the Data Integration Module 210, which initiates network communications with the pharmacy claims portion of the medical and pharmacy claims system 285. In some embodiments, the pharmacy claims data may reside in a separate data store from medical claims, requiring the Data Integration Module 210 to establish a second connection. In other embodiments, the medical and pharmacy claims system 285 may provide unified access to both medical and pharmacy claims through a single connection, enabling the Data Integration Module 210 to reuse the connection established in step 320. The Data Integration Module 210 may determine the appropriate connection approach by reading configuration parameters stored in the Data Repository 280.

At step 350, the current medication regimen and prescription fill history are extracted. This operation is performed by the Data Integration Module 210, which constructs and transmits pharmacy claims retrieval requests. In some embodiments, the Data Integration Module 210 may construct a query specifying the patient identifiers and a pharmacy-specific lookback period, such as one hundred eighty days, selected to capture recent prescription activity indicative of currently active medications. The returned pharmacy claim records may contain medication identifiers, fill dates specifying when prescriptions were dispensed, quantity dispensed values, and days supply values specifying the intended duration of the dispensed quantity. The Data Integration Module 210 may determine the current medication regimen by analyzing the pharmacy claim records using a medication currency algorithm. The medication currency algorithm may calculate an expected exhaustion date for each prescription by adding the days supply value to the fill date, compare the expected exhaustion date to the current date, and classify medications with expected exhaustion dates in the future or within a configurable grace period as currently active medications. The Data Integration Module 210 may compile the prescription fill history by sorting pharmacy claim records chronologically and grouping records by medication identifier to create a time-ordered sequence of dispensing events for each medication.

At step 360, a connection is established with the electronic health record system 287. This operation is performed by the Data Integration Module 210, which initiates network communications using healthcare interoperability protocols. In some embodiments, the Data Integration Module 210 may establish the connection using Fast Healthcare Interoperability Resources (FHIR) APIs or Health Level Seven (HL7) messaging protocols depending on the capabilities of the electronic health record system 287. The connection establishment may involve registering the Data Integration Module 210 as an authorized application, obtaining authorization credentials, and negotiating data format parameters. The Data Integration Module 210 may adapt its connection approach based on the specific electronic health record system implementation by loading configuration parameters that specify the appropriate protocol and authentication mechanism for the target system.

At step 370, chronic condition indicators and comorbidity data are extracted. This operation is performed by the Data Integration Module 210, which queries the electronic health record system 287 for problem list data. In some embodiments, the Data Integration Module 210 may query for active problem list entries representing documented chronic conditions currently managed as part of the patient's ongoing care. The returned data may contain condition codes and clinical status indicators specifying whether each condition is active or resolved. The chronic condition indicators may identify persistent health conditions such as asthma, chronic obstructive pulmonary disease (COPD), congestive heart failure, coronary artery disease, diabetes mellitus, hypertension, or chronic kidney disease that may increase patient vulnerability to adverse environmental conditions. The Data Integration Module 210 may parse the returned data by extracting the condition codes and mapping the extracted codes to a standardized chronic condition indicator set comprising boolean flags indicating the presence or absence of each relevant chronic condition. The Data Integration Module 210 may derive comorbidity data by counting the number of chronic condition indicators set to true for each patient, generating a comorbidity count value, and identifying specific comorbidity combinations known to increase environmental vulnerability, such as the co-occurrence of COPD and congestive heart failure.

At step 380, a connection is established with the care management system 288. This operation is performed by the Data Integration Module 210, which initiates network communications with the care management system 288 via the network 190. In some embodiments, the care management system 288 may be a module within the electronic health record system 287, enabling the Data Integration Module 210 to reuse the connection established in step 360. In other embodiments, the care management system 288 may be a standalone platform requiring separate connection establishment. The Data Integration Module 210 may authenticate with the care management system 288 using credentials appropriate to the system configuration.

At step 390, care management encounter records are extracted. This operation is performed by the Data Integration Module 210, which queries the care management system 288 for patient encounter history. In some embodiments, the care management encounter records may comprise documentation of prior interactions between care managers and the patient, including telephonic outreach attempts, care plan discussions, and coordination of services. The encounter records may contain encounter dates, encounter types distinguishing telephonic, in-person, and electronic encounters, and encounter dispositions indicating whether patients were reached and engaged. The Data Integration Module 210 may analyze the encounter history to calculate a patient engagement score by determining the ratio of successful contacts to attempted contacts, enabling subsequent outreach channel selection by the Orchestration Engine 240. The patient engagement score may be stored as part of the patient-level health data.

At step 395, patient-level health data is compiled and transmitted to the Risk Prediction Engine 230. This operation is performed by the Data Integration Module 210, which aggregates the data extracted in steps 330, 350, 370, and 390 into a unified patient-level health data structure. In some embodiments, the Data Integration Module 210 may perform data normalization operations by mapping diagnosis codes from different coding systems to a common terminology and standardizing date formats across data sources. The Data Integration Module 210 may construct a patient-level health data object for each patient containing the medical history comprising the aggregated diagnosis codes and claim records, the current medication regimen comprising the list of currently active medications identified by the medication currency algorithm, the chronic condition indicators comprising the boolean flags derived from problem list data, the comorbidity indicator comprising the comorbidity count and identified comorbidity combinations, the prescription fill history, and the care management encounter records comprising the encounter history and patient engagement score. The Data Integration Module 210 may transmit the compiled patient-level health data to the Risk Prediction Engine 230 via an internal message queue. In some embodiments, the Data Integration Module 210 may store the compiled patient-level health data in the Data Repository 280 via the Database Engine 270 to enable caching and reduce the need for repeated extraction from external systems.

In some embodiments, the operations of steps 310 through 395 may occur automatically when the Environmental Data Module 220 detects forecasted environmental threshold exceedances, while in other embodiments the operations may be initiated on-demand by healthcare administrators through the Communication and User Interface Module 265. The automated execution of these data extraction steps enables the patient risk management platform to compile comprehensive patient health profiles from multiple data sources within timeframes that would be impractical using manual data gathering processes. In some embodiments, the Data Integration Module 210 may execute extraction operations for multiple patients concurrently by spawning parallel processing threads, enabling efficient processing of large patient populations.

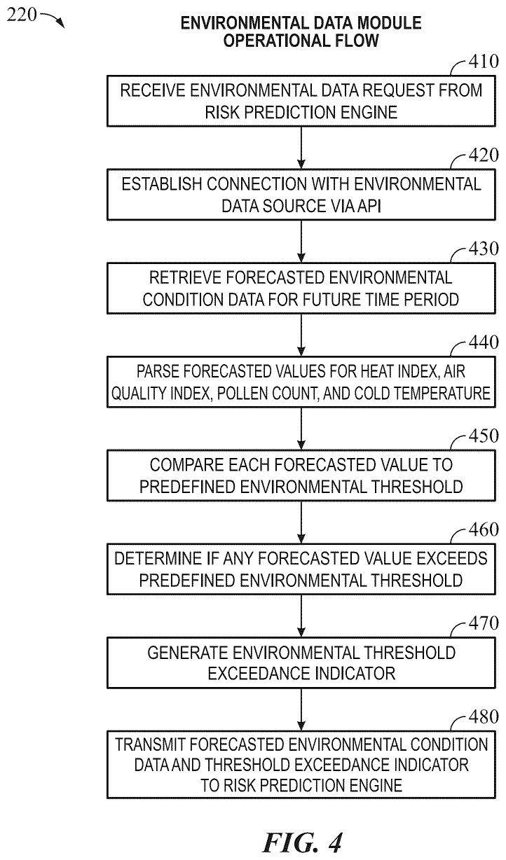

FIG. 4 is a flow diagram illustrating an exemplary operational sequence of the Environmental Data Module 220 in accordance with certain embodiments. The sequence shown may be implemented as computer-executable instructions within the application program 200 operating on computing system 100 of FIG. 2, with specific operations performed by the modules depicted therein. FIG. 4 illustrates the process by which the Environmental Data Module 220 retrieves forecasted environmental condition data, parses individual environmental parameter values, compares the parsed values to predefined thresholds, and generates threshold exceedance indicators for transmission to the Risk Prediction Engine 230.

At step 410, an environmental data request is received from the Risk Prediction Engine 230. This operation may be performed by the Environmental Data Module 220, which monitors an inbound message queue for environmental data requests. In some embodiments, the environmental data request may contain a geographic region specification comprising geographic identifiers such as postal codes, county codes, or latitude-longitude coordinate pairs defining the region for which environmental forecasts are requested. The request may further contain a forecast time horizon specifying the number of hours or days into the future for which forecasted environmental data should be retrieved, such as seventy-two hours or seven days. The Environmental Data Module 220 may parse the received request by extracting the geographic region specification and forecast time horizon and validating that the specified region falls within the service area covered by configured environmental data sources. In some embodiments, the Environmental Data Module 220 may initiate environmental data retrieval proactively based on a configured polling schedule rather than waiting for explicit requests, periodically querying environmental data sources to detect emerging environmental threshold exceedances.

At step 420, a connection is established with the environmental data source 290 via an API. This operation is performed by the Environmental Data Module 220, which initiates network communications with the environmental data source 290 via the network 190. In some embodiments, the environmental data source 290 may comprise a governmental weather data source providing forecast data, a governmental air quality monitoring data source providing air quality forecasts, or a third-party environmental data vendor providing aggregated environmental forecasts. The Environmental Data Module 220 may establish connections with multiple environmental data sources to retrieve different environmental parameter types, establishing separate connections for weather forecasts and air quality forecasts. The Environmental Data Module 220 may authenticate with each environmental data source by including an API key in request headers. In some embodiments, the Environmental Data Module 220 may implement connection pooling by maintaining persistent connections to frequently accessed environmental data sources, reducing connection establishment overhead for repeated queries.

At step 430, forecasted environmental condition data for a future time period is retrieved. This operation is performed by the Environmental Data Module 220, which transmits forecast retrieval requests to the environmental data source 290 and processes the returned forecast data. In some embodiments, the Environmental Data Module 220 may request weather forecast data specifying the geographic coordinates derived from the geographic region specification received in step 410. The weather data source may return a forecast response containing an array of forecast periods, wherein each forecast period represents a discrete time interval such as a three-hour window. Each forecast period may contain forecasted values for temperature, relative humidity, and derived indices such as heat index and wind chill. The Environmental Data Module 220 may request air quality forecast data specifying the geographic region. The air quality data source may return forecast data containing air quality index (AQI) values for multiple pollutant types and an overall AQI value representing the highest individual pollutant index. In some embodiments, the Environmental Data Module 220 may retrieve pollen forecast data from a third-party environmental data vendor, wherein the pollen forecast contains pollen count values for tree pollen, grass pollen, and weed pollen categories. The Environmental Data Module 220 may aggregate the forecast data retrieved from multiple sources into a unified forecasted environmental condition data structure indexed by geographic region and forecast time period.

At step 440, forecasted values for heat index, air quality index, pollen count, and cold temperature are parsed. This operation is performed by the Environmental Data Module 220, which extracts individual environmental parameter values from the aggregated forecast data retrieved in step 430. In some embodiments, the Environmental Data Module 220 may parse heat index values by iterating through the forecast periods, extracting the heat index value from each period, and identifying the maximum heat index value forecasted within the forecast time horizon. The heat index value represents the apparent temperature accounting for the combined effects of air temperature and relative humidity. The Environmental Data Module 220 may parse cold temperature values by extracting the minimum temperature value forecasted within the forecast time horizon, particularly during overnight periods. The Environmental Data Module 220 may parse air quality index values by extracting the overall AQI value from each forecast period and identifying the maximum AQI value forecasted within the forecast time horizon. The Environmental Data Module 220 may parse pollen count values by extracting the total pollen count or the maximum category-specific pollen count from the pollen forecast data. The parsed environmental parameter values may be stored in a structured data object containing the parameter type, the parsed value, the forecast time period, and the geographic region.

At step 450, each forecasted value is compared to a predefined environmental threshold. This operation is performed by the Environmental Data Module 220, which retrieves threshold values from the Data Repository 280 and executes numerical comparison operations. In some embodiments, the Environmental Data Module 220 may retrieve predefined environmental thresholds by querying the Data Repository 280 for threshold configuration records associated with the geographic region. Each threshold configuration record may contain an environmental parameter type identifier, a threshold value, and a threshold type indicator specifying whether the threshold represents a maximum value not to be exceeded or a minimum value not to fall below. For heat index values, the Environmental Data Module 220 may evaluate whether the parsed value exceeds the predefined heat index threshold, such as one hundred five degrees Fahrenheit. For air quality index values, the Environmental Data Module 220 may evaluate whether the parsed value exceeds the predefined AQI threshold, such as one hundred fifty representing unhealthy conditions. For pollen count values, the Environmental Data Module 220 may evaluate whether the parsed value exceeds the predefined pollen threshold representing high pollen conditions. For cold temperature values, the Environmental Data Module 220 may evaluate whether the parsed value falls below the predefined cold temperature threshold, such as thirty-two degrees Fahrenheit. The Environmental Data Module 220 may store the comparison results in a threshold comparison results array containing the parameter type, the forecasted value, the threshold value, and a boolean exceedance indicator for each comparison.

At step 460, a determination is made regarding whether any forecasted value exceeds the predefined environmental threshold. This operation is performed by the Environmental Data Module 220, which evaluates the threshold comparison results generated in step 450. In some embodiments, the Environmental Data Module 220 may iterate through the threshold comparison results array and evaluate the boolean exceedance indicator for each comparison result. If any exceedance indicator is set to true, the Environmental Data Module 220 may determine that a threshold exceedance exists and proceed to generate an environmental threshold exceedance indicator. The Environmental Data Module 220 may identify all environmental parameters for which threshold exceedances were detected. In some embodiments, the Environmental Data Module 220 may calculate an exceedance severity value for each threshold exceedance by computing the difference between the forecasted value and the threshold value, enabling prioritization of response activities based on the magnitude of forecasted environmental conditions.

At step 470, an environmental threshold exceedance indicator is generated. This operation is performed by the Environmental Data Module 220, which constructs a structured data object documenting the detected threshold exceedances. In some embodiments, the environmental threshold exceedance indicator may comprise an array of exceedance records, wherein each exceedance record contains the environmental parameter type for which an exceedance was detected, the forecasted value that exceeded the threshold, the threshold value that was exceeded, the exceedance severity value, the onset time at which the forecasted environmental condition is predicted to first exceed the threshold, the duration specifying how long the threshold exceedance is expected to persist, and the geographic region affected. The Environmental Data Module 220 may determine the onset time by analyzing the time-indexed forecast data to identify the earliest forecast period in which the environmental parameter value exceeds the threshold. The Environmental Data Module 220 may determine the duration by counting the consecutive forecast periods during which the threshold exceedance persists.

At step 480, forecasted environmental condition data and the threshold exceedance indicator are transmitted to the Risk Prediction Engine 230. This operation is performed by the Environmental Data Module 220, which packages the forecast data and exceedance indicators for transmission. In some embodiments, the Environmental Data Module 220 may construct an environmental data response message containing the forecasted environmental condition data comprising the parsed values for heat index, air quality index, pollen count, and cold temperature, and the environmental threshold exceedance indicator comprising the exceedance records generated in step 470. The Environmental Data Module 220 may transmit the message to the Risk Prediction Engine 230 via an internal message queue. The transmitted message may include the geographic region identifier and forecast time horizon from the original request. In some embodiments, the Environmental Data Module 220 may store the forecasted environmental condition data and threshold exceedance indicators in the Data Repository 280 via the Database Engine 270, creating a historical record for subsequent analysis.

In some embodiments, the operations of steps 410 through 480 may be executed on a continuous polling schedule, with the Environmental Data Module 220 periodically retrieving updated forecast data and re-evaluating threshold exceedances as forecasts are revised. In other embodiments, the Environmental Data Module 220 may subscribe to push notifications from environmental data sources that alert the system when significant forecast changes occur. The automated execution of these environmental data retrieval and threshold comparison steps enables the patient risk management platform to detect forecasted environmental threshold exceedances and initiate proactive patient outreach before adverse conditions materialize.

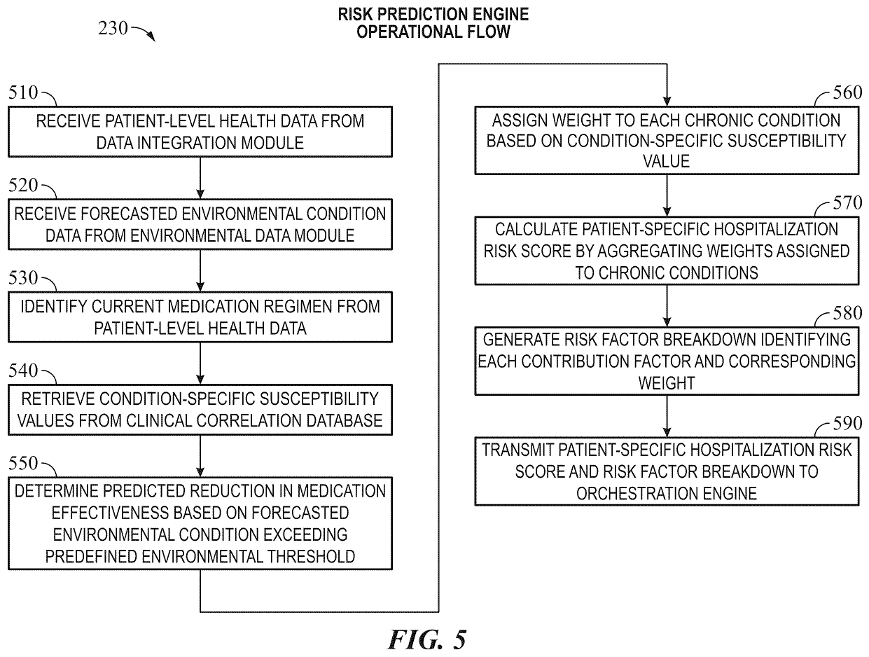

FIG. 5 is a flow diagram illustrating an exemplary operational sequence of the Risk Prediction Engine 230 in accordance with certain embodiments. The sequence shown may be implemented as computer-executable instructions within the application program 200 operating on computing system 100 of FIG. 2, with specific operations performed by the modules depicted therein. FIG. 5 illustrates the process by which the Risk Prediction Engine 230 correlates patient-level health data with forecasted environmental condition data, determines predicted reductions in medication effectiveness, assigns weights to chronic conditions based on susceptibility values, calculates patient-specific hospitalization risk scores, and generates risk factor breakdowns for transmission to the Orchestration Engine 240.

At step 510, patient-level health data is received from the Data Integration Module 210. This operation may be performed by the Risk Prediction Engine 230, which monitors an inbound message queue for patient data messages. In some embodiments, the patient-level health data may be received as an array of patient health data objects, wherein each object contains the compiled data for a single patient as described in step 395 of FIG. 3. Each patient health data object may contain the patient identifier, the medical history comprising diagnosis codes and claim records, the current medication regimen comprising currently active medications, the chronic condition indicators, the comorbidity indicator, the prescription fill history, and the care management encounter records. The Risk Prediction Engine 230 may index the received patient health data objects by patient identifier to enable efficient lookup during subsequent processing steps.

At step 520, forecasted environmental condition data is received from the Environmental Data Module 220. This operation is performed by the Risk Prediction Engine 230, which receives the environmental data response message transmitted in step 480 of FIG. 4. In some embodiments, the Risk Prediction Engine 230 may extract the forecasted environmental condition data comprising the parsed values for heat index, air quality index, pollen count, and cold temperature, and the environmental threshold exceedance indicator identifying which environmental parameters are forecasted to exceed thresholds. The Risk Prediction Engine 230 may extract the onset time from the environmental threshold exceedance indicator specifying when the forecasted environmental conditions are predicted to first exceed thresholds. The Risk Prediction Engine 230 may store the received environmental data in association with the geographic region identifier, enabling correlation with patient-level health data for patients residing within the affected region.

At step 530, the current medication regimen is identified from the patient-level health data. This operation is performed by the Risk Prediction Engine 230, which extracts and analyzes medication data for each patient. In some embodiments, the Risk Prediction Engine 230 may iterate through the patient health data objects received in step 510 and extract the current medication regimen array from each object. The current medication regimen may contain medication records comprising a medication identifier, medication name, therapeutic class code identifying the drug category, dosage form, and dosage strength. The Risk Prediction Engine 230 may classify each medication by mapping the therapeutic class code to a standardized medication category relevant to environmental health risk assessment. Relevant medication categories may include bronchodilators used to treat asthma and COPD, diuretics used to treat hypertension and heart failure, beta-blockers used to treat cardiovascular conditions, anticholinergic medications that may impair thermoregulation, and antidepressant medications that may increase heat sensitivity. The Risk Prediction Engine 230 may store the classified medication regimen for each patient for use in subsequent medication effectiveness analysis.

At step 540, condition-specific susceptibility values are retrieved from the clinical correlation database 292. This operation is performed by the Risk Prediction Engine 230, which queries the clinical correlation database 292 via the network 190. In some embodiments, the Risk Prediction Engine 230 may construct a query specifying the chronic condition codes identified across the patient population and the environmental parameter types for which threshold exceedances were detected. The clinical correlation database 292 may store condition-specific susceptibility values derived from historical hospitalization data correlating chronic conditions with hospitalization occurrences during prior environmental events. Each susceptibility value may quantify the correlation between a specific chronic condition and an increased hospitalization likelihood when exposed to specific environmental conditions. The susceptibility value may be expressed as a relative risk ratio, wherein a value of 2.0 indicates that patients with the specified chronic condition experienced hospitalizations at twice the rate of patients without the condition during prior environmental events. The Risk Prediction Engine 230 may parse the returned result set and store the susceptibility values in a lookup table indexed by chronic condition code and environmental parameter type.

At step 550, predicted reduction in medication effectiveness is determined based on forecasted environmental conditions exceeding the predefined environmental threshold. This operation is performed by the Risk Prediction Engine 230, which analyzes the interaction between patient medication regimens and forecasted environmental conditions. In some embodiments, the Risk Prediction Engine 230 may query the clinical correlation database 292 for medication-environment interaction records specifying how specific medication categories are affected by specific environmental conditions. Each medication-environment interaction record may contain a medication category identifier, an environmental parameter type, a threshold value above which the interaction effect becomes clinically significant, and an effectiveness reduction factor quantifying the expected reduction in medication effectiveness. The Risk Prediction Engine 230 may iterate through each patient's classified medication regimen, identify medications belonging to categories with documented environmental interactions, and determine whether the forecasted environmental conditions exceed the interaction threshold values. The predicted reduction in medication effectiveness may comprise reduced bronchodilator effectiveness during high heat index conditions, wherein elevated temperatures may reduce the delivery efficiency of inhaled medications. The predicted reduction in medication effectiveness may comprise increased adverse reaction risk for patients on antidepressant medications during high heat index conditions, wherein certain antidepressants may impair the body's thermoregulatory response to heat stress. The predicted reduction in medication effectiveness may comprise reduced inhaler effectiveness during high air quality index conditions, wherein elevated particulate matter may trigger increased airway inflammation that counteracts bronchodilator effects. The Risk Prediction Engine 230 may store the predicted medication effectiveness reductions for each patient in association with the affected medications and the environmental conditions triggering the reductions.

At step 560, a weight is assigned to each chronic condition based on the condition-specific susceptibility value. This operation is performed by the Risk Prediction Engine 230, which applies the susceptibility values retrieved in step 540 to the chronic conditions identified in each patient's health data. In some embodiments, the Risk Prediction Engine 230 may iterate through each patient health data object and examine the chronic condition indicators to identify which chronic conditions are present. For each chronic condition present in the patient-level health data, the Risk Prediction Engine 230 may retrieve the corresponding condition-specific susceptibility value from the lookup table constructed in step 540. The retrieved susceptibility value may serve as the base weight for the chronic condition. The Risk Prediction Engine 230 may apply weight adjustment factors to account for condition severity, comorbidity interactions, and medication effectiveness reductions. The severity adjustment factor may increase weights for chronic conditions documented as severe or uncontrolled based on recent exacerbation history. The comorbidity adjustment factor may increase weights when specific comorbidity combinations are present that compound environmental vulnerability. The medication adjustment factor may increase weights when predicted medication effectiveness reductions identified in step 550 affect medications used to manage the chronic condition.

In some embodiments, the Risk Prediction Engine 230 may assign the weight to each chronic condition by using the condition-specific susceptibility value as a base weight and applying multiplicative adjustment factors. For example, if the clinical correlation database 292 returns a susceptibility value of 2.5 for COPD during high heat index conditions, the Risk Prediction Engine 230 may use 2.5 as the base weight for COPD. The Risk Prediction Engine 230 may then apply a severity adjustment factor, such as 1.3 for severe or uncontrolled conditions, by multiplying the base weight by the adjustment factor to yield an adjusted weight of 3.25. The Risk Prediction Engine 230 may apply a comorbidity adjustment factor when the patient has multiple interacting chronic conditions, such as multiplying by 1.2 when COPD co-occurs with congestive heart failure, yielding a further adjusted weight of 3.9. The Risk Prediction Engine 230 may apply a medication adjustment factor when predicted medication effectiveness reductions affect medications managing the chronic condition, such as multiplying by 1.15 when inhaler effectiveness is predicted to be reduced, yielding a final adjusted weight of 4.485 for the COPD chronic condition. In other embodiments, the Risk Prediction Engine 230 may generate the weights and the patient-specific hospitalization risk scores using machine learning methods, wherein a machine learning model is trained on historical patient health data, environmental condition data, and hospitalization outcome data to predict hospitalization likelihood for patients based on their chronic conditions, medication regimens, and forecasted environmental exposures.

At step 570, the patient-specific hospitalization risk score is calculated by aggregating weights assigned to chronic conditions. This operation is performed by the Risk Prediction Engine 230, which combines the weighted chronic condition values into a single risk score for each patient. In some embodiments, the Risk Prediction Engine 230 may calculate the patient-specific hospitalization risk score by summing the adjusted weights assigned to each chronic condition present in the patient-level health data. The aggregation may apply a summation function that adds the weight values for all chronic conditions identified for the patient. In some embodiments, the Risk Prediction Engine 230 may apply a normalization function to scale the aggregated weight sum to a standardized risk score range, such as zero to one hundred, enabling consistent interpretation and threshold comparison across patients with varying numbers of chronic conditions. The Risk Prediction Engine 230 may apply a maximum score cap to prevent extreme outlier scores for patients with numerous severe chronic conditions. The calculated patient-specific hospitalization risk score may represent the relative likelihood that the patient will experience hospitalization when exposed to the forecasted environmental conditions compared to a baseline population.

In some embodiments, the Risk Prediction Engine 230 may calculate the patient-specific hospitalization risk score by summing the final adjusted weights for all chronic conditions present in the patient-level health data. For a patient with COPD having a final adjusted weight of 4.485, diabetes mellitus having a final adjusted weight of 1.8, and hypertension having a final adjusted weight of 1.2, the Risk Prediction Engine 230 may calculate the raw aggregated score as 7.485 by adding 4.485 plus 1.8 plus 1.2. The Risk Prediction Engine 230 may normalize the raw aggregated score to a standardized scale, such as zero to one hundred, by applying a normalization function that divides the raw score by a maximum expected score value and multiplies by one hundred. If the maximum expected score value is configured as 15.0, the normalized patient-specific hospitalization risk score would be calculated as 49.9 by dividing 7.485 by 15.0 and multiplying by one hundred.

At step 580, a risk factor breakdown is generated identifying each contributing factor and corresponding weight. This operation is performed by the Risk Prediction Engine 230, which compiles an itemized summary of the factors contributing to each patient's risk score. In some embodiments, the risk factor breakdown may comprise an itemized list containing an entry for each chronic condition, medication, and environmental vulnerability that contributed to the patient-specific hospitalization risk score. Each entry in the risk factor breakdown may contain the factor type identifying whether the factor is a chronic condition, medication interaction, or comorbidity combination, the factor name providing a human-readable description, and the corresponding weight assigned to the factor. The risk factor breakdown may further identify which specific environmental parameters triggered the risk assessment, such as high heat index or high air quality index, and the forecasted values for those parameters. The risk factor breakdown enables the Automated Outreach Module 250 to deliver personalized health guidance that addresses each patient's specific risk factors during outreach communications.

At step 590, the patient-specific hospitalization risk score and risk factor breakdown are transmitted to the Orchestration Engine 240. This operation is performed by the Risk Prediction Engine 230, which packages the calculated risk data for transmission. In some embodiments, the Risk Prediction Engine 230 may construct a risk assessment results message containing the patient identifier, the patient-specific hospitalization risk score, the risk factor breakdown, and the environmental threshold exceedance data received from the Environmental Data Module 220. The Risk Prediction Engine 230 may transmit the risk assessment results to the Orchestration Engine 240 via an internal message queue for generation of outreach target lists. In some embodiments, the Risk Prediction Engine 230 may store the calculated risk scores and risk factor breakdowns in the Data Repository 280 via the Database Engine 270 to enable historical tracking, trend analysis, and reporting on patient risk levels over time.

In some embodiments, the operations of steps 510 through 590 may be executed for each patient in the patient population affected by the forecasted environmental threshold exceedance. The Risk Prediction Engine 230 may process multiple patients concurrently by distributing risk calculation operations across parallel processing threads. The automated execution of these risk prediction steps enables the patient risk management platform to calculate patient-specific hospitalization risk scores incorporating medication effectiveness considerations that would be impractical to evaluate manually for large patient populations.

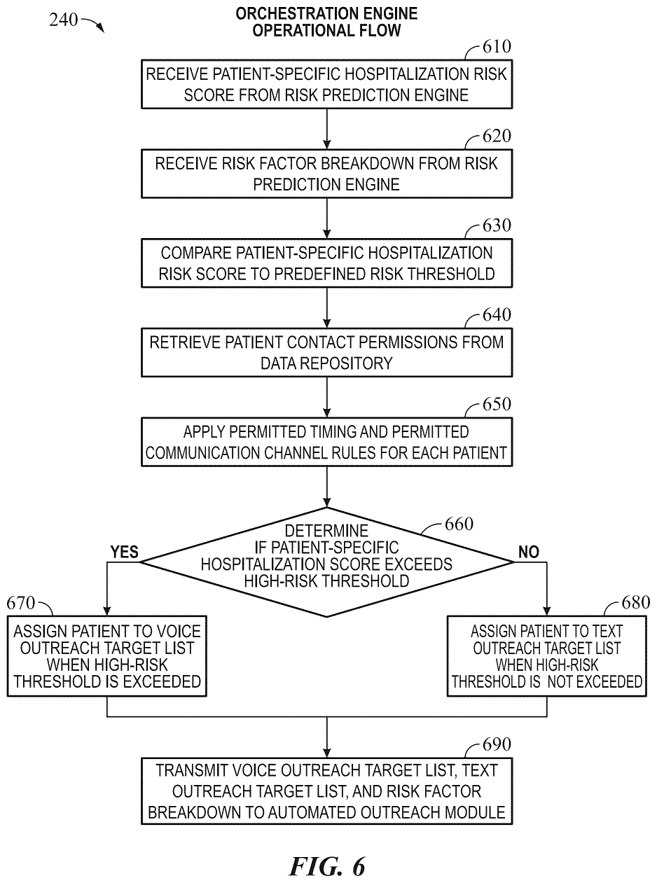

FIG. 6 is a flow diagram illustrating an exemplary operational sequence of the Orchestration Engine 240 in accordance with certain embodiments. The sequence shown may be implemented as computer-executable instructions within the application program 200 operating on computing system 100 of FIG. 2, with specific operations performed by the modules depicted therein. FIG. 6 illustrates the process by which the Orchestration Engine 240 receives risk assessment results, applies risk thresholds and patient contact permissions, and generates differentiated outreach target lists for transmission to the Automated Outreach Module 250.

At step 610, a patient-specific hospitalization risk score is received from the Risk Prediction Engine 230. This operation may be performed by the Orchestration Engine 240, which monitors an inbound message queue for risk assessment results transmitted by the Risk Prediction Engine 230. In some embodiments, the Orchestration Engine 240 may receive risk assessment results for multiple patients in a batch, wherein each result contains a patient identifier and the corresponding patient-specific hospitalization risk score calculated in step 570 of FIG. 5. The Orchestration Engine 240 may parse the received results and load the patient-specific hospitalization risk scores into a local data structure indexed by patient identifier for processing in subsequent steps.

At step 620, a risk factor breakdown is received from the Risk Prediction Engine 230. This operation is performed by the Orchestration Engine 240, which extracts the risk factor breakdown data from the risk assessment results message. In some embodiments, the risk factor breakdown may be included in the same message as the patient-specific hospitalization risk score, or may be transmitted in a separate message associated with the same patient identifier. The risk factor breakdown comprises the itemized list identifying each contributing factor to the patient-specific hospitalization risk score and the corresponding weight assigned to each factor as generated in step 580 of FIG. 5. The Orchestration Engine 240 may store the risk factor breakdown in association with the patient identifier for transmission to the Automated Outreach Module 250, enabling the AI agents to deliver personalized health guidance addressing each patient's specific risk factors.

At step 630, the patient-specific hospitalization risk score is compared to a predefined risk threshold. This operation is performed by the Orchestration Engine 240, which retrieves threshold configuration data and executes numerical comparison operations. In some embodiments, the Orchestration Engine 240 may retrieve the predefined risk threshold from the Data Repository 280 via the Database Engine 270. The predefined risk threshold may be configured by healthcare administrators through the Communication and User Interface Module 265 and may specify a minimum risk score value above which patients are considered candidates for proactive outreach. The Orchestration Engine 240 may execute a numerical comparison operation that evaluates whether each patient-specific hospitalization risk score exceeds the predefined risk threshold. Patients with risk scores below the predefined risk threshold may be excluded from outreach target lists, as their risk level does not warrant proactive intervention for the forecasted environmental event. The Orchestration Engine 240 may generate a filtered patient set containing only patients whose risk scores exceed the predefined risk threshold for further processing in subsequent steps.

In some embodiments, the predefined risk threshold may be configured as a normalized risk score value such as 25.0, indicating that patients with patient-specific hospitalization risk scores of 25.0 or higher are candidates for proactive outreach. The high-risk threshold applied in step 660 may be configured as a higher normalized risk score value such as 50.0, indicating that patients with scores of 50.0 or higher warrant voice-based outreach while patients with scores between 25.0 and 49.9 receive text-based outreach. Healthcare administrators may configure these threshold values through the Communication and User Interface Module 265 based on organizational risk tolerance, outreach capacity, and patient population characteristics.

At step 640, patient contact permissions are retrieved from the Data Repository 280. This operation is performed by the Orchestration Engine 240, which queries the Data Repository 280 for contact permission records associated with patients in the filtered patient set. In some embodiments, the patient contact permissions may define permitted timing and permitted communication channels for contacting each patient. The Orchestration Engine 240 may construct a database query specifying the patient identifiers in the filtered patient set and retrieve contact permission records containing the permitted contact time window, the preferred communication channel, the contact frequency limitation, and the do-not-contact indicator for each patient. The permitted contact time window may specify the hours during which the patient may be contacted, such as between 9:00 AM and 8:00 PM local time. The preferred communication channel may indicate whether the patient prefers voice calls or text messages. The contact frequency limitation may specify the maximum number of outreach attempts permitted within a specified time period. The do-not-contact indicator may specify that the patient has opted out of automated outreach communications. The Orchestration Engine 240 may store the retrieved patient contact permissions in association with patient identifiers for application in subsequent steps.

At step 650, permitted timing and permitted communication channel rules are applied for each patient. This operation is performed by the Orchestration Engine 240, which evaluates the patient contact permissions against the planned outreach timing and channel assignments. In some embodiments, the Orchestration Engine 240 may determine the planned outreach time based on the onset time extracted from the environmental threshold exceedance indicator and a predefined lead interval specifying how far in advance of the onset time outreach should be initiated. The Orchestration Engine 240 may evaluate whether the planned outreach time falls within the permitted contact time window for each patient. Patients whose permitted contact time window does not include the planned outreach time may have their outreach scheduled for the next available time within their permitted window, or may be flagged for manual follow-up if the permitted window does not occur before the environmental threshold exceedance onset. The Orchestration Engine 240 may further evaluate the preferred communication channel for each patient, noting preferences that may influence channel assignment in subsequent steps. Patients with a do-not-contact indicator set to true may be removed from the outreach candidate list and flagged for alternative notification methods such as provider notification or caregiver contact.

At step 660, a determination is made regarding whether the patient-specific hospitalization risk score exceeds a high-risk threshold. This operation is performed by the Orchestration Engine 240, which applies a secondary threshold comparison to differentiate between patients requiring voice outreach and patients suitable for text outreach. In some embodiments, the high-risk threshold may be configured as a value higher than the predefined risk threshold applied in step 630, creating a tiered risk classification system. The Orchestration Engine 240 may retrieve the high-risk threshold from the Data Repository 280 and execute a numerical comparison operation that evaluates whether each patient-specific hospitalization risk score in the filtered patient set exceeds the high-risk threshold. The comparison result determines the branching path for each patient, directing patients to either the voice outreach path or the text outreach path based on risk severity.

At step 670, patients are assigned to the voice outreach target list when the high-risk threshold is exceeded. This operation is performed by the Orchestration Engine 240 for patients whose patient-specific hospitalization risk score exceeds the high-risk threshold as determined in step 660. In some embodiments, the Orchestration Engine 240 may add the patient identifier to the voice outreach target list along with associated data including the patient contact information, the patient-specific hospitalization risk score, and the risk factor breakdown. The voice outreach target list may comprise an array of patient records, wherein each record contains the information needed by the Automated Outreach Module 250 to initiate voice-based outreach to the patient. Assignment to the voice outreach target list indicates that the patient's elevated risk level warrants more intensive engagement through spoken dialogue with the voice artificial intelligence agent. In some embodiments, the Orchestration Engine 240 may override the preferred communication channel specified in the patient contact permissions when the patient-specific hospitalization risk score exceeds the high-risk threshold, prioritizing voice outreach for highest-risk patients regardless of stated text preference.

At step 680, patients are assigned to the text outreach target list when the high-risk threshold is not exceeded. This operation is performed by the Orchestration Engine 240 for patients whose patient-specific hospitalization risk score does not exceed the high-risk threshold as determined in step 660. In some embodiments, the Orchestration Engine 240 may add the patient identifier to the text outreach target list along with associated data including the patient contact information, the patient-specific hospitalization risk score, and the risk factor breakdown. The text outreach target list may comprise an array of patient records formatted for text-based outreach. Assignment to the text outreach target list indicates that the patient's moderate risk level is appropriate for engagement through text-based messaging with the text artificial intelligence agent. In some embodiments, the Orchestration Engine 240 may evaluate the preferred communication channel for patients assigned to the text outreach target list and reassign patients with a strong voice preference to the voice outreach target list even when their risk score does not exceed the high-risk threshold, balancing risk-based channel assignment with patient preferences.

At step 690, the voice outreach target list, text outreach target list, and risk factor breakdown are transmitted to the Automated Outreach Module 250. This operation is performed by the Orchestration Engine 240, which packages the outreach target data for transmission. In some embodiments, the Orchestration Engine 240 may construct an outreach request message containing the voice outreach target list comprising patient records for voice-based outreach, the text outreach target list comprising patient records for text-based outreach, and the risk factor breakdowns for all patients on both lists. The outreach request message may further contain the onset time extracted from the environmental threshold exceedance indicator, enabling the Automated Outreach Module 250 to calculate appropriate outreach timing. The Orchestration Engine 240 may transmit the outreach request message to the Automated Outreach Module 250 via an internal message queue. In some embodiments, the Orchestration Engine 240 may store the generated outreach target lists in the Data Repository 280 via the Database Engine 270 to maintain a record of which patients were targeted for outreach during each environmental event.

In some embodiments, the operations of steps 610 through 690 may be executed whenever the Risk Prediction Engine 230 completes risk assessment for a patient population affected by forecasted environmental threshold exceedances. The Orchestration Engine 240 may process risk assessment results for large patient populations by iterating through patient records and applying threshold comparisons and contact permission rules efficiently. The automated execution of these orchestration steps enables the patient risk management platform to generate differentiated outreach target lists that balance risk severity with patient preferences and regulatory requirements.

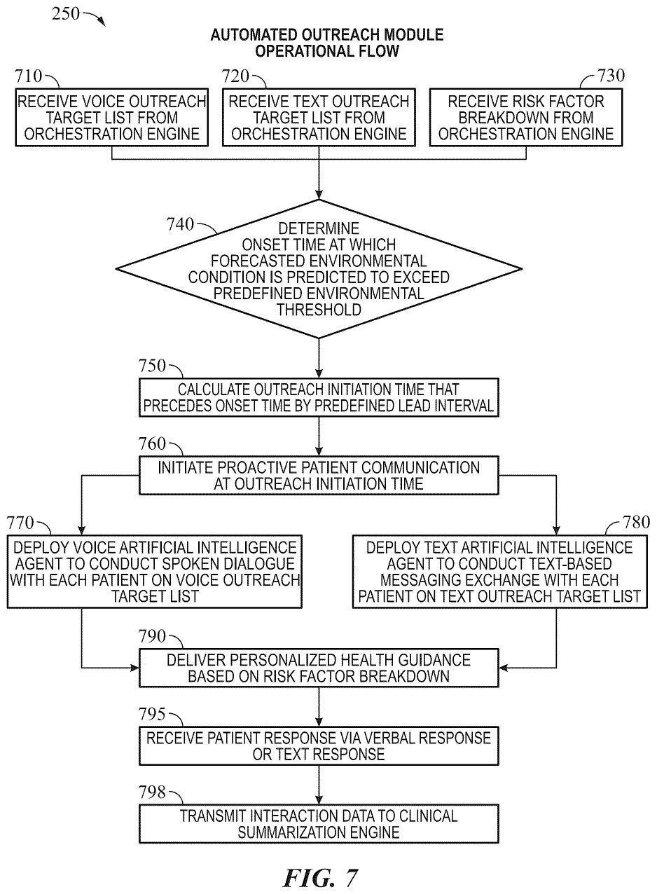

FIG. 7 is a flow diagram illustrating an exemplary operational sequence of the Automated Outreach Module 250 in accordance with certain embodiments. The sequence shown may be implemented as computer-executable instructions within the application program 200 operating on computing system 100 of FIG. 2, with specific operations performed by the modules depicted therein. FIG. 7 illustrates the process by which the Automated Outreach Module 250 receives outreach target lists, calculates outreach timing, deploys AI agents to conduct patient communications, delivers personalized health guidance, and captures patient responses for transmission to the Clinical Summarization Engine 260.

At step 710, the voice outreach target list is received from the Orchestration Engine 240. This operation may be performed by the Automated Outreach Module 250, which monitors an inbound message queue for outreach request messages. In some embodiments, the Automated Outreach Module 250 may extract the voice outreach target list from the outreach request message transmitted in step 690 of FIG. 6. The voice outreach target list may contain patient records for patients assigned to voice-based outreach, wherein each record includes the patient identifier, patient contact telephone number, patient-specific hospitalization risk score, and associated risk factor breakdown. The Automated Outreach Module 250 may load the voice outreach target list into a local data structure for processing by the voice artificial intelligence agent.

At step 720, the text outreach target list is received from the Orchestration Engine 240. This operation is performed by the Automated Outreach Module 250, which extracts the text outreach target list from the outreach request message. In some embodiments, the text outreach target list may contain patient records for patients assigned to text-based outreach, wherein each record includes the patient identifier, patient contact mobile number, patient-specific hospitalization risk score, and associated risk factor breakdown. The Automated Outreach Module 250 may load the text outreach target list into a local data structure for processing by the text artificial intelligence agent.

At step 730, the risk factor breakdown is received from the Orchestration Engine 240. This operation is performed by the Automated Outreach Module 250, which extracts the risk factor breakdown data associated with each patient on the outreach target lists. In some embodiments, the risk factor breakdown for each patient may be embedded within the patient record on the respective outreach target list, or may be transmitted as a separate data structure indexed by patient identifier. The Automated Outreach Module 250 may store the risk factor breakdowns in association with patient identifiers, enabling retrieval during personalized health guidance generation in step 790.

At step 740, an onset time is determined at which the forecasted environmental condition is predicted to exceed the predefined environmental threshold. This operation is performed by the Automated Outreach Module 250, which extracts timing information from the environmental threshold exceedance data included in the outreach request message. In some embodiments, the onset time may have been calculated by the Environmental Data Module 220 in step 470 of FIG. 4 and passed through the Risk Prediction Engine 230 and Orchestration Engine 240 to the Automated Outreach Module 250. The onset time specifies the date and time at which the forecasted environmental parameter value is predicted to first exceed the predefined environmental threshold. The Automated Outreach Module 250 may parse the onset time from the message data and convert the onset time to a standard datetime format for use in outreach timing calculations.

At step 750, an outreach initiation time is calculated that precedes the onset time by a predefined lead interval. This operation is performed by the Automated Outreach Module 250, which applies a time calculation to determine when outreach communications should begin. In some embodiments, the Automated Outreach Module 250 may retrieve the predefined lead interval from the Data Repository 280, wherein the predefined lead interval specifies the number of hours or days before the onset time that outreach should be initiated. The predefined lead interval may be configured by healthcare administrators to ensure that patients receive health guidance with sufficient time to take preventive actions, such as arranging transportation to a cooling center or adjusting medication timing. The Automated Outreach Module 250 may calculate the outreach initiation time by subtracting the predefined lead interval from the onset time. For example, if the onset time is 2:00 PM on a given date and the predefined lead interval is twenty-four hours, the outreach initiation time would be calculated as 2:00 PM on the preceding day. The Automated Outreach Module 250 may further adjust the calculated outreach initiation time to comply with permitted contact time windows specified in patient contact permissions, advancing or delaying outreach for individual patients as needed.

At step 760, proactive patient communication is initiated at the outreach initiation time. This operation is performed by the Automated Outreach Module 250, which monitors the system clock and triggers outreach activities when the current time reaches the calculated outreach initiation time. In some embodiments, the Automated Outreach Module 250 may implement a scheduling mechanism that queues outreach activities for execution at the outreach initiation time. When the outreach initiation time arrives, the Automated Outreach Module 250 may activate the voice artificial intelligence agent and text artificial intelligence agent to begin processing their respective outreach target lists. The proactive patient communication may be initiated such that each patient on the voice outreach target list and each patient on the text outreach target list receives personalized health guidance prior to the onset time when the forecasted environmental conditions are predicted to exceed the predefined environmental threshold.

At step 770, a voice artificial intelligence agent is deployed to conduct spoken dialogue with each patient on the voice outreach target list. This operation is performed by the Automated Outreach Module 250, which activates the voice artificial intelligence agent and provides the voice outreach target list for processing. In some embodiments, the voice artificial intelligence agent may be configured to conduct a spoken dialogue with the patient to deliver the personalized health guidance and to receive a verbal response from the patient. The voice artificial intelligence agent may initiate outbound telephone calls to patient devices 146 associated with patients on the voice outreach target list by interfacing with a telephony system via the network 190. For each patient on the voice outreach target list, the voice artificial intelligence agent may dial the patient contact telephone number, wait for the call to be answered, and upon connection begin the spoken dialogue. The voice artificial intelligence agent may use speech synthesis technology to convert text-based health guidance messages into spoken audio delivered to the patient through the telephone connection. The voice artificial intelligence agent may use speech recognition technology to convert the patient's spoken responses into text for processing and storage.

At step 780, a text artificial intelligence agent is deployed to conduct text-based messaging exchange with each patient on the text outreach target list. This operation is performed by the Automated Outreach Module 250, which activates the text artificial intelligence agent and provides the text outreach target list for processing. In some embodiments, the text artificial intelligence agent may be configured to conduct a text-based messaging exchange with the patient to deliver the personalized health guidance and to receive a text response from the patient. The text artificial intelligence agent may transmit text messages to patient devices 146 associated with patients on the text outreach target list by interfacing with a messaging gateway via the network 190. For each patient on the text outreach target list, the text artificial intelligence agent may compose a text message containing the personalized health guidance and transmit the message to the patient contact mobile number. The text artificial intelligence agent may monitor for incoming text message responses from patients and process received responses for storage and analysis.

At step 790, personalized health guidance is delivered based on the risk factor breakdown. This operation is performed by the voice artificial intelligence agent and text artificial intelligence agent, which generate patient-specific health guidance content using the risk factor breakdown data. In some embodiments, the personalized health guidance may comprise at least one of educational information regarding the forecasted environmental condition data, a recommended preventive action based on the current medication regimen, or a location of a resource facility. The AI agents may retrieve the risk factor breakdown for each patient and generate health guidance content that addresses the specific chronic conditions, medication considerations, and environmental vulnerabilities identified in the breakdown. For a patient with COPD and reduced inhaler effectiveness during high air quality conditions, the personalized health guidance may include information about the forecasted air quality deterioration, a recommendation to use the inhaler preventively before outdoor exposure, and the location of an air-conditioned facility where the patient can seek refuge. For a patient on antidepressant medication with increased adverse reaction risk during high heat conditions, the personalized health guidance may include information about the forecasted heat event, a recommendation to increase fluid intake and avoid prolonged outdoor exposure, and the location of a cooling center. The AI agents may adapt the language and detail level of the health guidance based on patient characteristics such as health literacy level or preferred language if such information is available in the patient contact permissions.

At step 795, a patient response is received via verbal response or text response. This operation is performed by the voice artificial intelligence agent and text artificial intelligence agent, which capture and process patient responses to the delivered health guidance. In some embodiments, the voice artificial intelligence agent may receive a verbal response from the patient through the telephone connection, apply speech recognition to convert the verbal response to text, and analyze the response content to determine the patient's acknowledgment, questions, or concerns. The text artificial intelligence agent may receive a text response from the patient through the messaging gateway and analyze the response content. The AI agents may engage in multi-turn dialogue with patients, answering questions about the health guidance, providing additional information about resource facility locations, or addressing patient concerns about medication adjustments. The AI agents may classify patient responses into categories such as acknowledged, declined, requested callback, expressed confusion, or no response to inform follow-up action recommendations. In some embodiments, patients expressing distress, confusion, or urgent health concerns may be flagged for immediate escalation to a human care manager.

At step 798, interaction data is transmitted to the Clinical Summarization Engine 260. This operation is performed by the Automated Outreach Module 250, which packages the communication records for transmission. In some embodiments, the interaction data may comprise a record for each patient communication attempt, wherein each record contains the patient identifier, the outreach target list assignment indicating voice or text channel, the communication timestamp, the health guidance content delivered, the patient response content received, and the response classification. The Automated Outreach Module 250 may construct an interaction data message containing records for all patient communications conducted during the outreach campaign and transmit the message to the Clinical Summarization Engine 260 via an internal message queue.

In some embodiments, the operations of steps 710 through 798 may be executed concurrently for multiple patients, with the voice artificial intelligence agent and text artificial intelligence agent processing their respective outreach target lists in parallel. The Automated Outreach Module 250 may manage concurrent outreach activities by allocating telephony resources for voice calls and messaging throughput for text messages based on available system capacity. The automated execution of these outreach steps enables the patient risk management platform to conduct proactive patient communications at scale, reaching potentially thousands of at-risk patients within the timeframe preceding forecasted environmental threshold exceedances.

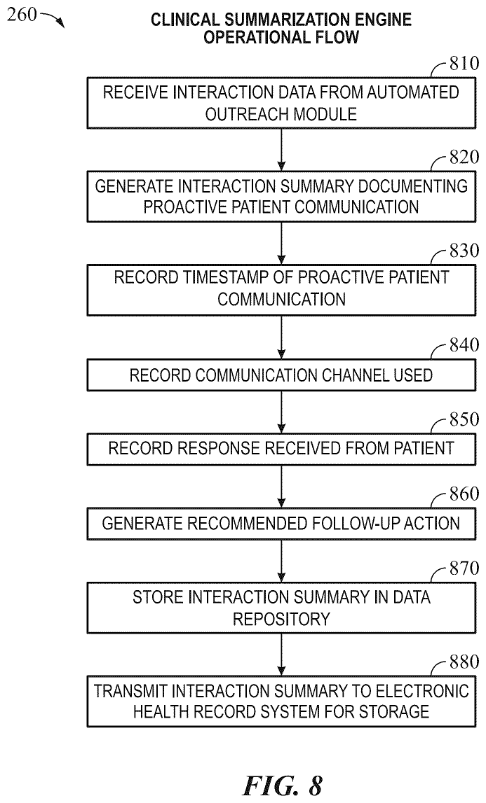

FIG. 8 is a flow diagram illustrating an exemplary operational sequence of the Clinical Summarization Engine 260 in accordance with certain embodiments. The sequence shown may be implemented as computer-executable instructions within the application program 200 operating on computing system 100 of FIG. 2, with specific operations performed by the modules depicted therein. FIG. 8 illustrates the process by which the Clinical Summarization Engine 260 receives interaction data, generates interaction summaries documenting proactive patient communications, and stores the summaries in both the Data Repository 280 and the electronic health record system 287.

At step 810, interaction data is received from the Automated Outreach Module 250. This operation may be performed by the Clinical Summarization Engine 260, which monitors an inbound message queue for interaction data messages transmitted by the Automated Outreach Module 250 in step 798 of FIG. 7. In some embodiments, the interaction data may comprise records for multiple patient communications conducted during the outreach campaign. Each interaction record may contain the patient identifier, the communication channel used, the communication timestamp, the health guidance content delivered, the patient response content, and the response classification. The Clinical Summarization Engine 260 may parse the received interaction data and load the interaction records into a processing queue for summary generation.

At step 820, an interaction summary is generated documenting the proactive patient communication. This operation is performed by the Clinical Summarization Engine 260, which creates a structured summary document for each patient interaction. In some embodiments, the Clinical Summarization Engine 260 may generate the interaction summary by extracting relevant information from the interaction record and formatting the information into a clinical documentation structure suitable for storage in electronic health records. The interaction summary may be formatted according to clinical documentation standards used by the healthcare organization, enabling seamless integration with existing care coordination workflows. The Clinical Summarization Engine 260 may apply natural language generation techniques to produce human-readable summary text that describes the outreach interaction in clinical terminology.

At step 830, the timestamp of the proactive patient communication is recorded. This operation is performed by the Clinical Summarization Engine 260, which extracts and formats the communication timestamp. In some embodiments, the Clinical Summarization Engine 260 may extract the timestamp from the interaction record indicating when the communication was initiated or completed. The timestamp may be formatted according to the date and time conventions used by the electronic health record system 287 to ensure consistent display and sorting when the interaction summary is stored in the patient's health record. The timestamp enables care teams to understand when the proactive outreach occurred relative to the forecasted environmental event and other care activities.

At step 840, the communication channel used is recorded. This operation is performed by the Clinical Summarization Engine 260, which documents whether the communication was conducted via voice call or text message. In some embodiments, the Clinical Summarization Engine 260 may record the communication channel by extracting the channel indicator from the interaction record, wherein the channel indicator specifies whether the patient was contacted by the voice artificial intelligence agent or the text artificial intelligence agent. The recorded communication channel enables care teams to understand the modality of the outreach interaction and may inform future outreach channel preferences for the patient.

At step 850, the response received from the patient is recorded. This operation is performed by the Clinical Summarization Engine 260, which documents the patient's response to the proactive outreach. In some embodiments, the Clinical Summarization Engine 260 may record the patient response by extracting the response content and response classification from the interaction record. The response content may comprise a transcription of the patient's verbal response for voice communications or the text of the patient's reply message for text communications. The response classification may indicate whether the patient acknowledged the health guidance, declined assistance, requested a callback, expressed confusion, or did not respond. Recording the patient response enables care teams to understand how the patient reacted to the proactive outreach and whether additional follow-up is warranted.

At step 860, a recommended follow-up action is generated. This operation is performed by the Clinical Summarization Engine 260, which analyzes the patient response and outreach outcome to determine appropriate next steps. In some embodiments, the Clinical Summarization Engine 260 may apply a decision logic that maps response classifications to recommended follow-up actions. For patients who acknowledged the health guidance and indicated understanding, the recommended follow-up action may be routine monitoring with no immediate action required. For patients who requested a callback or expressed confusion, the recommended follow-up action may be to schedule a care management call within a specified timeframe. For patients who expressed distress or health concerns, the recommended follow-up action may be immediate escalation to a human care manager or clinical staff. For patients who did not respond to the outreach attempt, the recommended follow-up action may be a repeat outreach attempt or alternative contact through a caregiver or emergency contact. The recommended follow-up action may be included in the interaction summary to guide care team activities.

At step 870, the interaction summary is stored in the Data Repository 280. This operation is performed by the Clinical Summarization Engine 260, which persists the generated interaction summary for internal record-keeping and analysis. In some embodiments, the Clinical Summarization Engine 260 may execute a database insert operation via the Database Engine 270 to store the interaction summary in the Data Repository 280. The stored interaction summary may be associated with the patient identifier, the environmental event identifier, and the outreach campaign identifier to enable retrieval and aggregation for reporting purposes. Storing interaction summaries in the Data Repository 280 enables the patient risk management platform to maintain a comprehensive record of outreach activities, track outreach effectiveness over time, and support quality improvement initiatives.

At step 880, the interaction summary is transmitted to the electronic health record system 287 for storage. This operation is performed by the Clinical Summarization Engine 260, which transmits the interaction summary to the external electronic health record system 287 via the network 190. In some embodiments, the Clinical Summarization Engine 260 may format the interaction summary according to healthcare interoperability standards such as HL7 or FHIR to ensure compatibility with the electronic health record system 287. The Clinical Summarization Engine 260 may transmit the formatted interaction summary to an interface endpoint exposed by the electronic health record system 287, which receives the summary and stores it as a clinical document in the patient's health record. Storing the interaction summary in the electronic health record system 287 enables care teams accessing the patient's record to view documentation of the proactive outreach, understand the health guidance delivered, review the patient's response, and act on recommended follow-up actions. The integration of interaction summaries into electronic health records supports care coordination by ensuring that all members of the patient's care team have visibility into proactive outreach activities.

In some embodiments, the operations of steps 810 through 880 may be executed for each patient interaction record received from the Automated Outreach Module 250. The Clinical Summarization Engine 260 may process multiple interaction records concurrently to generate and store interaction summaries efficiently. The automated execution of these summarization steps enables the patient risk management platform to document proactive outreach activities in clinical systems without requiring manual documentation effort from care managers or clinical staff.

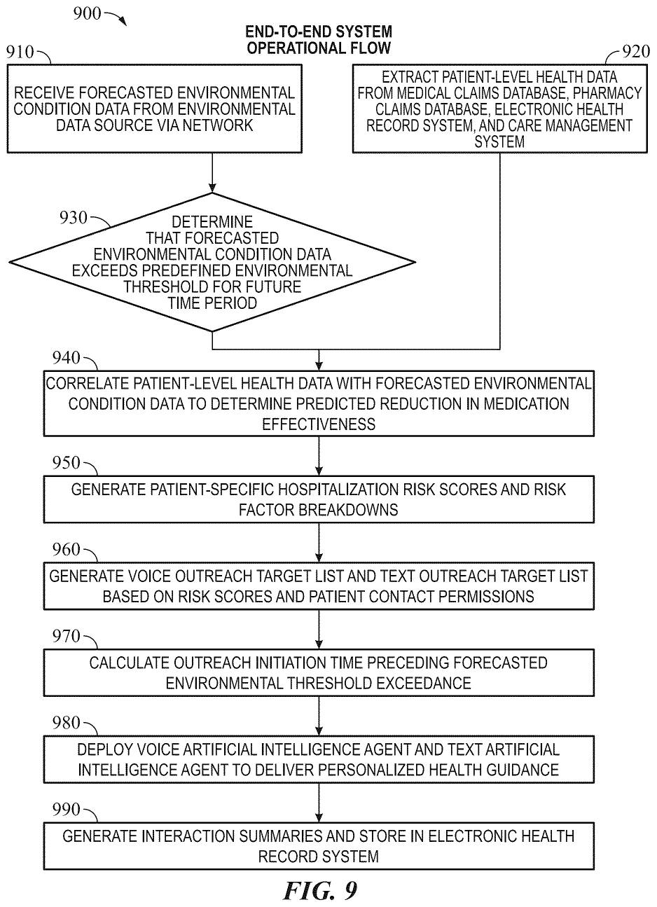

FIG. 9 is a flow diagram illustrating an exemplary end-to-end system operational flow 900 in accordance with certain embodiments. The sequence shown may be implemented as computer-executable instructions within the application program 200 operating on computing system 100 of FIG. 2, with operations distributed across the modules depicted therein. FIG. 9 illustrates the complete workflow from environmental data receipt through patient outreach and clinical documentation, demonstrating how the modules of the patient risk management platform coordinate to enable proactive hospitalization prevention.

At step 910, forecasted environmental condition data is received from the environmental data source 290 via the network 190. This operation may be performed by the Environmental Data Module 220, which retrieves forecasted values for environmental parameters as described in steps 410 through 430 of FIG. 4. In some embodiments, the Environmental Data Module 220 may receive forecasted environmental condition data on a scheduled polling basis or in response to push notifications from environmental data sources indicating significant forecast changes. The forecasted environmental condition data may comprise predicted values for heat index, air quality index, pollen count, and cold temperature for future time periods across geographic regions served by the healthcare organization.

At step 920, patient-level health data is extracted from the medical claims database, pharmacy claims database, electronic health record system 287, and care management system 288. This operation may be performed concurrently with step 910 by the Data Integration Module 210, which retrieves patient health information from multiple external systems as described in steps 320 through 390 of FIG. 3. In some embodiments, the Data Integration Module 210 may maintain cached patient-level health data in the Data Repository 280 and refresh the cached data on a periodic basis, enabling rapid risk assessment when environmental threshold exceedances are detected without requiring real-time extraction from external systems. The extracted patient-level health data may comprise medical history, current medication regimens, chronic condition indicators, comorbidity data, prescription fill history, and care management encounter records for patients residing in geographic regions covered by the environmental forecasts.

At step 930, a determination is made that the forecasted environmental condition data exceeds the predefined environmental threshold for a future time period. This operation is performed by the Environmental Data Module 220, which compares forecasted values to configured thresholds as described in steps 450 through 470 of FIG. 4. In some embodiments, step 930 represents a decision point that governs whether subsequent risk assessment and outreach activities are initiated. If the forecasted environmental condition data does not exceed any predefined environmental threshold, the system may continue monitoring for future forecast updates without initiating outreach activities. If the forecasted environmental condition data exceeds one or more predefined environmental thresholds, the system proceeds to step 940 to initiate risk assessment and outreach activities. The determination may identify which specific environmental parameters are forecasted to exceed thresholds, the magnitude of the exceedances, and the onset time when threshold exceedances are predicted to begin.

At step 940, patient-level health data is correlated with forecasted environmental condition data to determine predicted reduction in medication effectiveness. This operation is performed by the Risk Prediction Engine 230, which analyzes medication-environment interactions as described in steps 530 through 550 of FIG. 5. In some embodiments, the Risk Prediction Engine 230 may identify patients whose current medication regimens include medications known to have reduced effectiveness or increased adverse reaction risk under the forecasted environmental conditions. The correlation may involve querying the clinical correlation database 292 for medication-environment interaction records and evaluating whether forecasted conditions exceed the thresholds at which medication effectiveness is affected. The predicted reductions in medication effectiveness may inform weight adjustments applied during risk score calculation, increasing risk scores for patients whose medication effectiveness is expected to be compromised.

At step 950, patient-specific hospitalization risk scores and risk factor breakdowns are generated. This operation is performed by the Risk Prediction Engine 230, which calculates risk scores by aggregating weighted chronic condition values as described in steps 560 through 580 of FIG. 5. In some embodiments, the Risk Prediction Engine 230 may generate a patient-specific hospitalization risk score for each patient in the affected geographic region by retrieving condition-specific susceptibility values from the clinical correlation database 292, assigning weights to chronic conditions based on the susceptibility values, applying adjustment factors for condition severity, comorbidities, and medication effectiveness reductions, and aggregating the adjusted weights into a composite risk score. The Risk Prediction Engine 230 may generate a risk factor breakdown for each patient comprising an itemized list of contributing factors and corresponding weights, enabling personalized health guidance that addresses each patient's specific vulnerabilities.

At step 960, a voice outreach target list and a text outreach target list are generated based on risk scores and patient contact permissions. This operation is performed by the Orchestration Engine 240, which applies threshold comparisons and contact permission rules as described in steps 630 through 680 of FIG. 6. In some embodiments, the Orchestration Engine 240 may compare each patient-specific hospitalization risk score to the predefined risk threshold to identify patients warranting proactive outreach, apply patient contact permissions to ensure compliance with patient preferences and regulatory requirements, and compare risk scores to the high-risk threshold to differentiate between patients assigned to voice outreach and patients assigned to text outreach. The voice outreach target list may contain patients whose risk scores exceed the high-risk threshold, while the text outreach target list may contain patients whose risk scores exceed the predefined risk threshold but do not exceed the high-risk threshold.

At step 970, an outreach initiation time is calculated preceding the forecasted environmental threshold exceedance. This operation is performed by the Automated Outreach Module 250, which determines when outreach activities should begin as described in steps 740 through 750 of FIG. 7. In some embodiments, the Automated Outreach Module 250 may calculate the outreach initiation time by subtracting the predefined lead interval from the onset time when forecasted environmental conditions are predicted to first exceed the predefined environmental threshold. The calculated outreach initiation time ensures that patients receive personalized health guidance with sufficient advance notice to take preventive actions before adverse environmental conditions materialize.

At step 980, a voice artificial intelligence agent and a text artificial intelligence agent are deployed to deliver personalized health guidance. This operation is performed by the Automated Outreach Module 250, which activates the AI agents to conduct patient communications as described in steps 770 through 790 of FIG. 7. In some embodiments, the voice artificial intelligence agent may conduct spoken dialogues with patients on the voice outreach target list, delivering personalized health guidance based on the risk factor breakdown and receiving verbal responses. The text artificial intelligence agent may conduct text-based messaging exchanges with patients on the text outreach target list, delivering personalized health guidance and receiving text responses. The personalized health guidance may comprise educational information regarding the forecasted environmental conditions, recommended preventive actions based on each patient's medication regimen and chronic conditions, and locations of resource facilities such as cooling centers or air-conditioned public spaces.

At step 990, interaction summaries are generated and stored in the electronic health record system 287. This operation is performed by the Clinical Summarization Engine 260, which documents proactive outreach activities as described in steps 820 through 880 of FIG. 8. In some embodiments, the Clinical Summarization Engine 260 may generate an interaction summary for each patient communication comprising the timestamp, communication channel, patient response, and recommended follow-up action. The interaction summaries may be stored in both the Data Repository 280 for internal tracking and the electronic health record system 287 for care team visibility. Storage in the electronic health record system 287 enables care teams to review documentation of proactive outreach, understand the health guidance delivered to each patient, and act on recommended follow-up actions.

In some embodiments, the end-to-end operational flow illustrated in FIG. 9 may be executed automatically when the Environmental Data Module 220 detects forecasted environmental threshold exceedances, with minimal manual intervention required from healthcare administrators. The coordinated execution of operations across the Data Integration Module 210, Environmental Data Module 220, Risk Prediction Engine 230, Orchestration Engine 240, Automated Outreach Module 250, and Clinical Summarization Engine 260 enables the patient risk management platform to identify at-risk patients, generate personalized risk assessments, conduct proactive outreach at scale, and document interactions in clinical systems within timeframes that would be impractical using manual processes. The automated end-to-end workflow enables healthcare organizations to prevent hospitalizations by reaching vulnerable patients before adverse environmental conditions occur, rather than responding reactively after patients experience health deterioration.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A computer-implemented system for predicting patient hospitalization risk due to environmental factors and preventing hospitalization through automated patient outreach, the system comprising:
at least one computing device in operable communication with a network;
a server in operable communication with the at least one computing device over the network, the server configured to host a patient risk management platform comprising:
a data integration module configured to extract patient-level health data from at least one of a medical claims database, a pharmacy claims database, an electronic health record system, or a care management system, wherein the patient-level health data comprises at least one of a medical history, a current medication regimen, or a chronic condition indicator;
an environmental data module configured to receive forecasted environmental condition data from at least one environmental data source, wherein the forecasted environmental condition data comprises a predicted value for at least one of a heat index, an air quality index, a pollen count, or a cold temperature for a future time period;
a risk prediction engine configured to generate a patient-specific hospitalization risk score and a risk factor breakdown by correlating the patient-level health data with the forecasted environmental condition data, wherein the risk prediction engine is configured to determine a predicted reduction in medication effectiveness for the current medication regimen when the forecasted environmental condition data exceeds a predefined environmental threshold during the future time period;
an orchestration engine configured to generate a voice outreach target list and a text outreach target list by comparing the patient-specific hospitalization risk score to a predefined risk threshold and by applying patient contact permissions that define permitted timing and permitted communication channels for contacting each patient;
an automated outreach module configured to initiate proactive patient communication to each patient on the voice outreach target list via a voice artificial intelligence agent and to each patient on the text outreach target list via a text artificial intelligence agent, wherein the voice artificial intelligence agent and the text artificial intelligence agent are each configured to deliver personalized health guidance based on the risk factor breakdown; and
a clinical summarization engine configured to generate an interaction summary documenting the proactive patient communication and to store the interaction summary in the electronic health record system.

2. The system of claim 1, wherein the patient-level health data further comprises at least one of a diagnosis code, a procedure code, a prescription fill history, a comorbidity indicator, or a care management encounter record.

3. The system of claim 1, wherein the data integration module is configured to extract the patient-level health data via at least one of an application programming interface or a custom data extraction routine.

4. The system of claim 1, wherein the at least one environmental data source comprises at least one of a governmental weather data source, a governmental air quality monitoring data source, or a third-party environmental data vendor.

5. The system of claim 1, wherein the environmental data module is configured to continuously monitor the forecasted environmental condition data and to transmit updated forecasted environmental condition data to the risk prediction engine when the forecasted environmental condition data changes.

6. The system of claim 1, wherein the predicted reduction in medication effectiveness comprises at least one of a reduced bronchodilator effectiveness during a high heat index condition, an increased adverse reaction risk for a patient on an antidepressant medication during a high heat index condition, or a reduced inhaler effectiveness during a high air quality index condition.

7. The system of claim 1, wherein the risk prediction engine is configured to identify a plurality of chronic conditions from the patient-level health data and to weight each of the plurality of chronic conditions based on a susceptibility of each chronic condition to the forecasted environmental condition data.

8. The system of claim 1, wherein the risk factor breakdown comprises an itemized list identifying each contributing factor to the patient-specific hospitalization risk score and a corresponding weight assigned to each contributing factor.

9. The system of claim 1, wherein the orchestration engine is configured to assign each patient to at least one of the voice outreach target list or the text outreach target list based on a severity level of the patient-specific hospitalization risk score, wherein patients having a patient-specific hospitalization risk score exceeding a high-risk threshold are assigned to the voice outreach target list.

10. The system of claim 1, wherein the patient contact permissions comprise at least one of a permitted contact time window, a preferred communication channel, a contact frequency limitation, or a do-not-contact indicator.

11. The system of claim 1, wherein the voice artificial intelligence agent is configured to conduct a spoken dialogue with the patient to deliver the personalized health guidance and to receive a verbal response from the patient, and wherein the text artificial intelligence agent is configured to conduct a text-based messaging exchange with the patient to deliver the personalized health guidance and to receive a text response from the patient.

12. The system of claim 1, wherein the personalized health guidance comprises at least one of educational information regarding the forecasted environmental condition data, a recommended preventive action based on the current medication regimen, or a location of a resource facility.

13. The system of claim 1, wherein the interaction summary comprises at least one of a timestamp of the proactive patient communication, a communication channel used, a response received from the patient, or a recommended follow-up action.

14. The system of claim 1, wherein the server comprises a cloud-based server configured to synchronize the patient-level health data and the forecasted environmental condition data across a plurality of healthcare facilities.

15. A computer-implemented method for predicting patient hospitalization risk due to environmental factors and preventing hospitalization through automated patient outreach, the method comprising:
extracting, via at least one processor, patient-level health data from at least one of a medical claims database, a pharmacy claims database, an electronic health record system, or a care management system, wherein the patient-level health data comprises at least one of a medical history, a current medication regimen, or a chronic condition indicator;
receiving, via the at least one processor, forecasted environmental condition data from at least one environmental data source, wherein the forecasted environmental condition data comprises a predicted value for at least one of a heat index, an air quality index, a pollen count, or a cold temperature for a future time period;
generating, via the at least one processor, a patient-specific hospitalization risk score and a risk factor breakdown by correlating the patient-level health data with the forecasted environmental condition data, wherein generating the patient-specific hospitalization risk score comprises determining a predicted reduction in medication effectiveness for the current medication regimen when the forecasted environmental condition data exceeds a predefined environmental threshold during the future time period;
generating, via the at least one processor, a voice outreach target list and a text outreach target list by comparing the patient-specific hospitalization risk score to a predefined risk threshold and by applying patient contact permissions that define permitted timing and permitted communication channels for contacting each patient;
initiating, via the at least one processor, proactive patient communication to each patient on the voice outreach target list via a voice artificial intelligence agent and to each patient on the text outreach target list via a text artificial intelligence agent, wherein the voice artificial intelligence agent and the text artificial intelligence agent each deliver personalized health guidance based on the risk factor breakdown;
generating, via the at least one processor, an interaction summary documenting the proactive patient communication; and
storing, via the at least one processor, the interaction summary in the electronic health record system.

16. The method of claim 15, further comprising continuously monitoring, via the at least one processor, the forecasted environmental condition data and updating the patient-specific hospitalization risk score when the forecasted environmental condition data changes during the future time period.

17. The method of claim 15, wherein generating the patient-specific hospitalization risk score further comprises:
identifying a plurality of chronic conditions from the patient-level health data;
retrieving, for each chronic condition of the plurality of chronic conditions, a condition-specific susceptibility value from a clinical correlation database, wherein the condition-specific susceptibility value is derived from historical hospitalization data correlating the chronic condition with hospitalization occurrences during prior environmental events;
assigning a weight to each chronic condition, wherein the weight corresponds to the condition-specific susceptibility value retrieved from the clinical correlation database; and
calculating the patient-specific hospitalization risk score by aggregating the weights assigned to each of the plurality of chronic conditions present in the patient-level health data.

18. The method of claim 15, wherein initiating proactive patient communication further comprises assigning each patient to at least one of the voice outreach target list or the text outreach target list based on a severity level of the patient-specific hospitalization risk score, wherein patients having a patient-specific hospitalization risk score exceeding a high-risk threshold are assigned to the voice outreach target list and patients having a patient-specific hospitalization risk score below the high-risk threshold are assigned to the text outreach target list.

19. A software product comprising at least one computer-readable storage medium having application instructions stored on the at least one computer-readable storage medium, the application instructions executable by at least one processor to:
extract patient-level health data from at least one of a medical claims database, a pharmacy claims database, an electronic health record system, or a care management system, wherein the patient-level health data comprises at least one of a medical history, a current medication regimen, or a chronic condition indicator;
receive forecasted environmental condition data from at least one environmental data source, wherein the forecasted environmental condition data comprises a predicted value for at least one of a heat index, an air quality index, a pollen count, or a cold temperature for a future time period;
generate a patient-specific hospitalization risk score and a risk factor breakdown by correlating the patient-level health data with the forecasted environmental condition data, wherein generating the patient-specific hospitalization risk score comprises determining a predicted reduction in medication effectiveness for the current medication regimen when the forecasted environmental condition data exceeds a predefined environmental threshold during the future time period;
generate a voice outreach target list and a text outreach target list by comparing the patient-specific hospitalization risk score to a predefined risk threshold and by applying patient contact permissions that define permitted timing and permitted communication channels for contacting each patient;
initiate proactive patient communication to each patient on the voice outreach target list via a voice artificial intelligence agent and to each patient on the text outreach target list via a text artificial intelligence agent, wherein the voice artificial intelligence agent and the text artificial intelligence agent each deliver personalized health guidance based on the risk factor breakdown;
generate an interaction summary documenting the proactive patient communication; and
store the interaction summary in the electronic health record system.

20. The software product of claim 19, wherein the application instructions are further executable to:
determine an onset time at which the forecasted environmental condition data is predicted to exceed the predefined environmental threshold;
calculate an outreach initiation time that precedes the onset time by a predefined lead interval; and
initiate the proactive patient communication at the outreach initiation time such that each patient on the voice outreach target list and each patient on the text outreach target list receives the personalized health guidance prior to the onset time.
